(19) United States
(12) Patent Application Publication
(10) Pub. No.: US 2021/0239706 A1
(43) Pub. Date: Aug. 5, 2021

(54) METHODS FOR WHOLE-CELL GLYCOPROTEOMIC ANALYSIS

(71) Applicant: Regeneron Pharmaceuticals, Inc., Tarrytown, NY (US)

(72) Inventors: Yuan MAO, Hartsdale, NY (US); Shruti NAYAK, Wood-Ridge, NJ (US)

(21) Appl. No.: 17/161,796

(22) Filed: Jan. 29, 2021

Related U.S. Application Data
(60) Provisional application No. 62/968,536, filed on Jan. 31, 2020.

(51) Int. Cl. G01N 33/68

(52) U.S. Cl. CPC G01N 33/6842; G01N 2458/15; G01N 33/6848

(57) ABSTRACT

The present disclosure relates to glycoproteomics. More specifically, the current disclosure provides methods for determining one or more of the glycoproteins, glycosylation sites, glycopeptide fragments, and glycan compositions of both membrane and cytosolic proteins. The methods herein employ a single processing method that enables extraction of membrane and cytosolic proteins for the identification and analysis of whole-cell glycosylation, independent of species or sample type.

### CROSS REFERENCE TO RELATED APPLICATIONS

This application claims the benefit of priority from U.S. Provisional Application No. 62/968,536, filed Jan. 31, 2020, the entire contents of which is incorporated herein by reference.

### FIELD OF THE DISCLOSURE

The present disclosure relates to glycoproteomics. More specifically, the current disclosure provides methods for determining the glycoprotein, glycosite, glycopeptide and glycan composition of both membrane and cytosolic proteins. The methods herein employ a single processing method that enables extraction of membrane and cytosolic proteins for the identification and quantitative analysis of whole-cell glycosylation.

### BACKGROUND

Complete genomic sequences and large partial sequence databases have the potential to identify every gene in a species. However, genetic code alone cannot explain biological and clinical processes because gene sequences alone fail to elucidate how the genes and their products (proteins) cooperate to carry out a specific biological processes or functions. Furthermore, a nucleotide sequence does not predict the amount or the activity of a gene's protein product(s) nor does it speak to modification of proteins. Therefore, to fully understand the physiological state or make up of cells or organisms quantitative analysis of proteins and their post-translational modifications are required.

Glycosylation is a well recognized post-translational modification, whereby glycans (i.e., oligosaccharide chains), are attached covalently attached to cellular proteins. Glycosylation occurs at specific locations along the polypeptide backbone of a protein. There are two primary types of glycosylation: glycosylation characterized by O-linked oligosaccharides, which are attached to serine or threonine residues; and glycosylation characterized by N-linked oligosaccharides, which are attached to asparagine residues in an Asn-X-Ser/Thr sequence, where X can be any amino acid except proline. Glycosylation is a diverse process that involves many intracellular components (e.g., the nucleus, cytosol, golgi and endoplasmic reticulum). For example, N-acetylneuramic acid (i.e., sialyl acid), which is a terminal residue of both N-linked and O-linked oligosaccharides is synthesized in the nucleus. Additionally, sugars and N-linked oligosaccharides are synthesized in the cytosol. However, N-linked and O-linked glycosylation of most proteins occurs in the endoplasmic reticulum (ER) and Golgi. See, e.g., Van Kooyk et al. Front. Immunol. (2013) 4:451.

Glycosylation affects the protein function, such as protein stability, enzymatic activity and protein-protein interactions. Therefore, glycosylation is a critical component of protein quality control and also serves important functional roles in mature membrane proteins, including involvement in adhesion and signaling. As such, most studies focus on the glycoproteomic analysis of membrane proteins alone.

Studies on glycosylation of membrane proteins have been complicated by the unique physical properties of membrane proteins, including the hydrophobicity of the transmembrane domain(s) of integral membrane proteins which frequently leads to aggregation and loss during isolation. Therefore, methods to profile and analyze the glycoproteins from both the cell membrane and cytosol are important to determine the complete glycoproteome, which would lead to more a more consistent and reproducible means for evaluating glycoproteins.

### SUMMARY OT THE DISCLOSURE

The present methods are based, in part, on the discovery that intact glycoproteins from both intracellular compartments (cytosol) and membrane(s) of cells can be efficiently and consistently isolated from complex cellular samples in a single process for use in mass-spectrometry based glycoproteomic analysis of the entire cell.

In one aspect of the present disclosure, a method for profiling of glycoproteins is provided that includes (a) processing a sample including cells in order to isolate a cytosolic fraction of proteins from the cells and a membrane fraction of proteins from the cells, and (b) performing a mass spectrometry analysis of the proteins in the membrane fraction to obtain a profile of glycoproteins in the membrane fraction, and performing a mass spectrometry analysis of the proteins in the cytosolic fraction to obtain a profile of glycoproteins in the cytosolic fraction.

In some embodiments, the sample is a sample comprising mammalian cells. In specific embodiments, the sample is a sample of human cells or a sample of murine cells. In one embodiment, the sample is a sample of human cells. In another embodiment, the sample is a sample of murine cells. In certain embodiments, the sample is comprised of adherent cells. In other embodiments, the sample is a suspension of cells. In yet another embodiment, the sample is a soft tissue sample including cells. In other embodiments, the sample is a hard tissue sample including cells.

In some embodiments, the methods include the use of liquid chromatography-mass spectrometry (LC-MS) to obtain a profile of glycoproteins in the membrane fraction and a profile of glycoproteins in the cytosolic fraction of cells.

In certain embodiments, the processing step of the method includes mixing the cells from the sample with a permeabilization solution comprising a first detergent to permeabilize the plasma membrane of the cells in the sample. In some embodiments, the permeabilization solution includes a first detergent that is mild enough to permeabilize the membranes of cells to permit the release of cytosolic proteins from cellular compartments but does not release transmembrane proteins from membranes. In certain embodiments, the permeabilization solution includes one or more nonionic detergents. In certain embodiments, the permeabilization solution comprises 0.1%-0.2% nonionic detergent. In specific embodiments, the nonionic detergent is, for example, Triton-X 100, octylphenoxypolyethoxyethanol (nonidet P-40, NP-40, IGEPAL CA-630), polysorbate 20 (Tween-20) or Saponin. In one instance, the permeabilization solution is the Permeabilization Buffer described in the Mem-PER™ Membrane Protein Extraction Kit (Thermo Scientific™), the entire contents of which is incorporated herein by reference.

In some embodiments, the method includes subjecting the mixture to centrifugation to obtain a first pellet of permeabilized cells, and a supernatant including the cytosolic fraction of proteins.

In further embodiments, the method includes collecting the supernatant composed of the cytosolic fraction of proteins, and suspending the first pellet of permeabilized cells in a solubilization solution including a second detergent to form a suspension including solubilized membrane proteins from the cells. In some embodiments, the solubilization solution includes a detergent that is capable of solubilizing membrane proteins from the permeabilized cells. In certain embodiments, the solubilization solution includes one or more ionic detergents. In some embodiments, the solubilization solution includes an ionic detergent at a concentration of 0.1% to 1.0% weight by volume. In specific embodiments, the ionic detergent is, for example, sodium dodecyl sulfate (SDS), sodium deoxycholate, N-lauryl sarcosine or 3-[(3-cholamidopropyl)dimethylammonio]-1-propanesulfonate (CHAPS). In one embodiment, the solubilization solution comprises SDS and sodium deoxycholate. In a specific embodiment, the solubilization solution includes SDS, sodium deoxycholate, and octylphenoxypolyethoxyethanol. In one instance, the solubilization solution is the Solubilization Buffer described in the Mem-PER™ Membrane Protein Extraction Kit (Thermo Scientific™), the entire contents of which is incorporated herein by reference.

In some instances, the method includes subjecting the suspension composed of soluble membrane proteins to centrifugation to obtain a (second) pellet and a supernatant comprising the membrane fraction of proteins, and collecting the supernatant.

In some embodiments, the profile of glycoproteins identified by mass spectrometry analysis of either the membrane fraction of proteins and/or cytosolic fraction of proteins from the cells is obtained by a process that includes digesting proteins in the membrane fraction to obtain a sample of peptide fragments from the membrane fraction and/or digesting proteins in the cytosolic fraction to obtain a sample of peptide fragments from the cytosolic fraction. In certain embodiments, the digestion is carried out by Filter Assisted Sample Preparation (FASP).

In embodiments, the method includes separating non-glycosylated peptide fragments from the samples of peptide fragments from the cytosolic fraction and/or the membrane fraction of proteins in order to obtain enriched glycosylated peptides from the membrane fraction of the cells and/or the cytosolic fraction of the cells. In some instances, the samples of peptide fragments from the cytosolic fraction and/or the membrane fraction of proteins are enriched by removing non-glycosylated peptides through ion-pairing hydrophilic interaction liquid chromatography (HILIC), lectin affinity chromatography, or hydrazide capture. In a specific embodiment, the sample of peptide fragments from the cytosolic fraction of proteins is enriched by ion-pairing HILIC. In another embodiment, the sample of peptide fragments from the membrane fraction of proteins is enriched by ion-pairing HILIC.

In some embodiments, the present methods include releasing the glycans from the enriched samples of glycoproteins or peptide fragments. In one embodiment, glycans are released from enriched sample of peptide fragments from the cytosolic fraction by contacting the sample with a glycosidase, such as an amidase. In another embodiment, glycans are released from enriched sample of glycopeptides fragments from the membrane fraction of proteins by contacting the sample with a glycosidase, such as an amidase.

In certain embodiments, the method of profiling glycoproteins includes performing a mass spectrometry analysis of the peptide fragments enriched in glycosylated peptides, to obtain the profile of glycoproteins in the membrane fraction and/or the profile of glycoproteins in the membrane fraction. In some embodiments, the glycoprotein profile identifies a listing of glycoproteins. In certain embodiments the glycoprotein profile identifies one or more of the following glycoprotein characteristics: a glycosylation site, glycopeptide quantity in a fraction, glycan composition, or abundance of the glycoproteins.

In further embodiments, the method of profiling glycoproteins includes obtaining the glycoproteomic profile of a cytosolic fraction of proteins and/or a membrane fraction of proteins by searching the mass spectra data from the cytosolic fraction of proteins and/or a membrane fraction of proteins against a proteome database. In some embodiments, the proteome database is the Uniprot human proteome database or the Uniprot mouse proteome database. In one embodiment, the sample of cells includes human cells and the mass spectra data from the cytosolic fraction of proteins and/or a membrane fraction of proteins is searched against the Uniprot human proteome database. In another embodiment, the sample of cells includes murine cells and the mass spectra data from the cytosolic fraction of proteins and/or a membrane fraction of proteins is searched against the Uniprot mouse proteome database.

In another embodiment, the method of profiling glycoproteins includes obtaining the glycoproteomic profile of a cytosolic fraction of proteins and/or a membrane fraction of proteins by searching the mass spectra data from the cytosolic fraction of proteins and/or a membrane fraction of proteins against a proteome database and a glycan database. In certain embodiments, the sample of cells includes human cells and the mass spectra data from the cytosolic fraction of proteins and/or a membrane fraction of proteins is searched against the Uniprot human proteome database and a human glycan database, such as Byonic™ human glycan database in order to identify the glycopeptides, PSM, glycoproteins, glycan composition and glycosylation sites in each fraction. In another embodiment, the sample of cells includes murine cells and the mass spectra data from the cytosolic fraction of proteins and/or a membrane fraction of proteins is searched against the Uniprot mouse proteome database and a murine glycan database such as, for example, the Byonic™ mammalian glycan database in order to identify the glycopeptides, PSM, glycoproteins, glycan composition and/or glycosylation sites in each fraction.

In yet another embodiment, the profile of glycoproteins in the cytoplasmic fraction and the profile of glycoproteins in the membrane fraction of cells obtained by the present methods are compared in order to obtain the unique number of glycosylation sites, glycopeptides, glycans, and/or glycoproteins in each fraction or in the whole-cell.

The present disclosure also recognizes that the inventive methods can be used to determine the variability in proteins across samples or across preparations of samples. For example, the inventors have shown that the present methods can be used to determine whether or not a variation in the protein production, protein location or post-translational modification of proteins exists across samples or preparations thereof.

Therefore, in another aspect of the present disclosure a method for detecting protein variation between samples or preparations of samples is provided. In one embodiment, the method for detecting protein variation includes (a) processing a first sample including cells in order to isolate a cytosolic fraction of proteins from the cells and a membrane fraction of proteins from the cells of the first sample, and (b) processing a second sample composed of cells in order to isolate a cytosolic fraction of proteins from the cells and a membrane fraction of proteins from the cells of the second sample, and (c) digesting the proteins in the cytosolic and membrane fractions in the first sample in order to obtain peptide fragments from the cytosolic fraction and the membrane fraction from the cells of the first sample, and (d) digesting the proteins in the cytosolic and membrane fractions in the second sample in order to obtain peptide fragments from the cytosolic fraction and the membrane fraction from the cells of the second sample, and (e) labeling the peptide fragments in the cytosolic fraction from the first sample (e.g., with a with a detectable marker) and labeling the peptide fragments in the cytosolic fraction from the second sample, and mixing the labeled cytosolic fractions to obtain a mixture of labeled cytosolic peptide fragments from the first and second samples, and (f) labeling the peptide fragments in the membrane fraction from the first sample and labeling the peptide fragments in the membrane fraction of cells from the second sample, mixing the labeled membrane fractions to obtain a mixture of labeled membrane peptide fragments from the first and second samples, and (g) detecting the cytosolic peptide fragments in the mixture of labeled cytosolic peptide fragments; and detecting the membrane peptide fragments in the mixture of labeled membrane peptide fragments, thereby determining whether or not any variation in the total amount of cytosolic proteins and/or membrane proteins exists between the first sample and the second sample.

In certain embodiments, processing the cells of the first and second sample includes mixing the cells from one of the samples with a permeabilization solution comprising a first detergent to permeabilize the plasma membrane of the cells in the sample. This processing will then be carried out on the cells of the other sample. In some embodiments, the permeabilization solution includes a first detergent that is mild enough to permeabilize the membranes of cells to permit the release of cytosolic proteins from cellular compartments but does not release transmembrane proteins from membranes. In certain embodiments, the permeabilization solution includes one or more nonionic detergents. In certain embodiments, the permeabilization solution comprises 0.1%-0.2% nonionic detergent. In specific embodiments, the nonionic detergent is, for example, Triton-X 100, octylphenoxypolyethoxyethanol (nonidet P-40, NP-40, IGEPAL CA-630), polysorbate 20 (Tween-20) or Saponin. In one instance, the permeabilization solution is the Permeabilization Buffer described in the Mem-PER™ Membrane Protein Extraction Kit (Thermo Scientific™), the entire contents of which is incorporated herein by reference.

In certain embodiments, the method includes subjecting each of the permeabilized mixtures (i.e., from each sample) to centrifugation to obtain a first pellet of permeabilized cells, and a supernatant including the cytosolic fraction of proteins.

In further embodiments, the method includes collecting the supernatant composed of the cytosolic fraction of proteins from each individual sample of cells and, separately, suspending each of the first pellets of permeabilized cells in a solubilization solution including a second detergent to form a suspension including solubilized membrane proteins from the cells. In some embodiments, the solubilization solution includes a detergent that is capable of solubilizing membrane proteins from the permeabilized cells. In certain embodiments, the solubilization solution includes one or more ionic detergents. In some embodiments, the solubilization solution includes an ionic detergent at a concentration of 0.1% to 1.0% weight by volume. In specific embodiments, the ionic detergent is, for example, sodium dodecyl sulfate (SDS), sodium deoxycholate, N-lauryl sarcosine or 3-[(3-cholamidopropyl)dimethylammonio]-1-propanesulfonate (CHAPS). In one embodiment, the solubilization solution comprises SDS and sodium deoxycholate. In a specific embodiment, the solubilization solution includes SDS, sodium deoxycholate, and octylphenoxypolyethoxyethanol. In one instance, the solubilization solution is the Solubilization Buffer described in the Mem-PER™ Membrane Protein Extraction Kit (Thermo Scientific™), the entire contents of which is incorporated herein by reference.

In some instances, the method includes subjecting each of the suspensions composed of soluble membrane proteins from each of the samples or sample preparations to centrifugation to obtain a set of (second) pellets and a set of supernatants comprising the membrane fraction of proteins from each of the samples, and collecting the supernatants.

As indicated above, the method for detecting protein variation between samples or preparations of samples includes labeling each fraction, such as with a detectable marker. In some instances, the detectable marker for each of the cytosolic fractions obtained from the first and second sample of cells are different. In certain embodiments, the detectable marker for each of the cytosolic fractions obtained from the first and second sample of cells (or preparations thereof) are the same. In other instances, the detectable marker for each of the membrane fractions obtained from the first and second sample of cells are different. In some instances, the detectable marker for each of the membrane fractions obtained from the first and second sample of cells are the same. In some embodiments, the detectable markers used to label peptide fragments in each cytosolic fraction are different from one another, and the same detectable markers are used to label peptide fragments in the membrane fraction of the first and second sample of cells, or preparations thereof. In specific embodiments, the detectable markers are used to label peptide fragments in each cytosolic fraction are the same as the detectable markers used to label peptide fragments in each membrane fraction.

In some embodiments, the detectable markers are isobaric detectable markers that covalently label primary amines (—NH2 groups) and lysine residues. In certain embodiments, the isobaric detectable marker contains heavy isotopes, which are detectable in mass spectrometry for sample identification and quantitation of peptides. In a specific embodiment, the proteins or peptides are labeled with isobaric detectable markers as described in the Thermo Scientific™ Tandem Mass Tag (TMT) system (Thermo Scientific™), the entire contents of which is incorporated herein by reference.

In various embodiments, the inventive methods include performing a mass spectrometry analysis of a mixture of labeled cytosolic peptides to obtain a profile of glycoproteins in the cytosolic fractions of the first and second samples (or preparations thereof), and performing a mass spectrometry analysis of a mixture of labeled membrane peptides to obtain a profile of glycoproteins in the membrane fractions of the first and second samples (or preparations thereof). In certain embodiments, mass spectrometry is performed on the mixture of labeled cytosolic peptide fragments to obtain the profile of glycoproteins in the cytosolic fractions of the first sample and the profile of glycoproteins in the cytosolic fraction of digested proteins the second sample, wherein each of said profiles comprise a listing of glycoproteins, optionally with one or more of glycosylation sites, glycopeptides, glycan composition, and abundance of the glycoproteins.

In other embodiments, the present methods include separating non-glycosylated peptide fragments from each of the mixtures of cytosolic peptide fragments to obtain a collection of cytosolic peptide fragments from the first sample and second sample enriched in glycosylated peptide fragments. In certain embodiments, non-glycosylated peptide fragments are separated from each of the mixtures of membrane peptide fragments to obtain a collection of membrane peptide fragments from the first sample and second sample enriched in glycosylated peptide fragments.

In some instances, the samples of peptide fragments from the mixture of cytosolic peptide fragments and/or the mixture of membrane peptide fragments are enriched by removing non-glycosylated peptides through ion-pairing hydrophilic interaction liquid chromatography (HILIC), lectin affinity chromatography, or hydrazide capture. In a specific embodiment, the mixture of cytosolic peptide fragments is enriched by ion-pairing HILIC. In another embodiment, the mixture of membrane peptide fragments of proteins is enriched by ion-pairing HILIC.

In some embodiments, the present methods include releasing the glycans from the enriched samples of glycoproteins or peptide fragments. In one embodiment, glycans are released from an enriched sample of peptides fragments from the mixture of cytosolic peptide fragments by contacting the mixture with a glycosidase, such as an amidase. In another embodiment, glycans are released from an enriched mixture of membrane peptide fragments by contacting the mixture with a glycosidase, such as an amidase.

### BRIEF DESCRIPTION OF DRAWINGS

FIGS. 1A-1B depict exemplary mass spectrometry analysis of glycoproteins from the membranes and cytosol of adherent cells. Adherent cells were grown to confluence and harvested. Harvested cells were processed according to the present methods and the proteins of the membrane fraction (A) and cytosolic fraction (B) were analyzed by liquid chromatography-mass spectrometry (LCMS). The mass spectrum observed for each glycopeptide fragment detected in the fractions analyzed (19-36) were compared to a human protein sequence database and a Byonic™ human glycan database to obtain a peptide spectrum match (PSM) for the glycopeptides present in each fraction.

FIGS. 2A-2D depict exemplary mass spectrometry analysis of glycoproteins from the membranes and cytosol of adherent cells to obtain whole-cell glycoprotein profile. (A) LCMS analysis of the total number of glycoproteins detected in the membrane fraction and cytosolic fraction of an adherent cell preparation reveals 307 glycoproteins that are unique to the membranes of cells, 49 glycoproteins that are unique to the cytosolic fraction of cells, and 180 glycoproteins found in both the cytoplasmic fraction and membrane fraction of an exemplary adherent cell sample. (B) LCMS analysis determined the total number of glycosylation sites (glycosites) detected in the membrane fraction and cytosolic fraction of an adherent cell preparation. 569 unique glycosites were identified in the membrane fraction of the cells, 40 unique glycosites were detected in the cytosolic fraction of the cells, and 325 unique glycosites were identified in proteins from both the membrane and cytosolic fractions. (C) LCMS detected 3641 unique glycopeptides in the membrane fraction of the cells, 348 unique glycopeptides in the cytosolic fraction of the processed cells and 1165 glycopeptide that were identified in both the cytosolic and membrane fractions of an exemplary adherent cell sample. (D) LCMS analysis identified 25 unique glycans from the membrane fraction of the cells, 1 unique glycan in the cytosolic fraction of the cells and 95 glycans in both the cytosolic fraction and membrane fraction of the adherent cell sample.

FIGS. 3A-3B depict a mass spectrometry analysis of glycoproteins from the membranes and cytosol of cells obtained from murine liver tissue samples. Soft liver tissue samples were obtained and processed according to the present methods to obtain a cytosolic fraction of proteins and a membrane fraction of proteins from each liver tissue sample. The proteins of the membrane fraction (A) and cytosolic fraction (B) were analyzed by LCMS. The mass spectra observed for each glycopeptide fragment detected in the fractions analyzed (19-36) were compared to a predicted mass spectrum database and a Byonic™ mammalian glycan database to identify the peptide spectrum match (PSM).

FIGS. 4A-4D depict a mass spectrometry analysis of glycoproteins from the membranes and cytosol of cells obtained from murine liver tissue samples to generate whole-cell glycoprotein profiles. (A) LCMS analysis of the total number of glycoproteins detected in the membrane fraction and cytosolic fraction of a liver tissue preparation reveals 212 glycoproteins that are unique to the membranes of hepatic cells, 89 glycoproteins that are unique to the cytosolic fraction of the hepatic cells, and 359 glycoproteins found in both the cytoplasmic fraction and membrane fraction of an liver tissue sample. (B) LCMS analysis determined the total number of glycosylation sites (glycosites) detected in the membrane fraction and cytosolic fraction of a cell preparation obtained from liver tissue. 555 unique glycosites were identified in the membrane fraction of the cells, 317 unique glycosites were detected in the cytosolic fraction of the cells, and 577 unique glycosites were identified in proteins from both the membrane and cytosolic fractions. (C) LCMS detected 331 unique glycopeptide fragments in the membrane fraction of the hepatic cells, 1592 unique glycopeptide fragments in the cytosolic fraction of the processed liver tissue cells, and 2646 glycopeptide fragments were identified in both the cytosolic and membrane fractions of the sample. (D) LCMS analysis identified 41 unique murine glycans from the membrane fraction of the liver cells, 20 unique glycans in the cytosolic fraction of the cells and 145 murine glycans in both the cytosolic fraction and membrane fraction of the liver tissue cell sample tested.

FIGS. 5A-5B depict the reproducibility of sample processing in replicate cytosolic fractions and membrane fractions obtained from human adherent cells. Liquid chromatography mass spectrometry is used to measure intensity of detectable marker generated signals (i.e., TMT reporter ions) generated in the HCD MS/MS spectra of (A) all proteins present in replicate preparations of membrane fractions from human K562 cells and (B) all proteins present in replicate preparations of cytosolic fractions from human K562 cells. The values plotted on the graph are Log 2 of marker signal intensity. Correlation coefficients (R2) of greater than 0.99 for each of the membrane and cytosolic preparations indicate that the processing methods for the isolation of cytosolic fractions and membrane fractions of peptides from adherent cells are highly consistent and reproducible.

FIGS. 6A-6B depict the reproducibility of sample processing in replicate cytosolic fractions and membrane fractions obtained from murine liver tissue. Liquid chromatography mass spectrometry is used to measure intensity of detectable marker generated signals (i.e., TMT reporter ions) generated in the HCD MS/MS spectra of (A) all proteins present in replicate preparations of membrane fractions from murine hepatic cells from soft liver tissue and (B) all proteins present in replicate preparations of cytosolic fractions from murine hepatic cells from soft liver tissue. The values plotted on the graph are Log 2 of marker signal intensity. Correlation coefficients (R2) of greater than 0.98 for each of the membrane and cytosolic preparations indicate that the processing methods for the isolation of cytosolic fractions and membrane fractions of peptides from soft tissue samples are highly consistent and reproducible.

### DETAILED DESCRIPTION

The inventors have developed a method for profiling glycosylation of proteins that are expressed in multiple cellular compartments, which identifies a holistic (whole-cell) profile of glycosylation in any biological system and enables quantitation of glycosylation.

Therefore, in one aspect of the present disclosure a method for profiling of glycoproteins is provided that includes a mass spectrometry-based proteomic analysis of a cytosolic fraction of proteins from a sample of cells and a mass spectrometry-based proteomic analysis of a membrane fraction of proteins from the cells to obtain a profile of glycoproteins in the cytosolic fraction, the membrane fraction, and whole-cell. More specifically, it has been demonstrated herein that intact glycoproteins from intracellular compartments (cytosol) and membrane(s) of cells can be efficiently and consistently isolated from complex cellular samples in a single process for use in mass-spectrometry based glycoproteomic analysis of the entire cell or individual fractions thereof.

The present methodology can be applied to many types of samples including, but not limited to, adherent samples of cells, cell suspensions, tissue samples (hard and soft), independent of the species of cell (e.g., human, mouse, avian, rat).

Through the use of the present methodology, the inventors also discovered that a sample of cells can be processed to obtain a cytosolic fraction of cells and a membrane fraction of cells from a first sample or sample preparation, and the protein concentrations in such cytosolic fractions and membrane fractions from the first sample or sample preparation can be compared to those obtained from a second sample or second preparation of the first sample to determine whether or not a variation in protein production, protein location or post-translational modification of proteins exists across samples or sample preparations.

### Definitions

As used herein, the following terms have the meanings indicated. As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Unless defined otherwise, all technical and scientific terms used herein have the same meaning as commonly understood by one of ordinary skill in the art to which this invention belongs. Still, certain terms are defined below for the sake of clarity and ease of reference.

By “peptide” is meant a short polymer formed from the linking individual amino acid residues together, where the link between one amino acid residue and the second amino acid residue is called an amide bond or a peptide bond. A peptide or peptide fragment comprises at least two amino acid residues. A peptide is distinguished from a polypeptide in that it is shorter. At least two peptides, linked together by an amide bond or peptide bond between the C′ terminal amino acid residue of one peptide and the N′ terminal amino acid residue of the second peptide, form a polypeptide in accordance with various embodiments of the invention.

By “polypeptide” or “protein” is meant a long polymer formed from the linking individual amino acid residue, where the link between one amino acid residue and the second amino acid residue is called an amide bond or a peptide bond. A polypeptide or protein comprises at least four amino acid residues; however, multiple polypeptides can be linked together via amide or peptide bonds to form an even longer protein. A peptide, polypeptide or protein can be modified by naturally occurring modifications such as post-translational modifications, including phosphorylation, fatty acylation, prenylation, sulfation, hydroxylation, acetylation, addition of carbohydrate, addition of prosthetic groups or cofactors, formation of disulfide bonds, proteolysis, assembly into macromolecular complexes, and the like.

A “peptide fragment” is a peptide of two or more amino acids, generally derived from a larger polypeptide or protein.

As used herein, a “glycopolypeptide”, “glycopeptide”, “glycosylated peptide”, “glycoprotein” or “glycosylated protein” refers to a peptide or polypeptide that contains a covalently bound carbohydrate group (a “glycan”). The carbohydrate or glycan can be a monosaccharide, oligosaccharide or polysaccharide. Proteoglycans are included within the above meaning. A glycopolypeptide, glycosylated polypeptide, glycoprotein, or glycosylated protein can additionally contain other post-translational modifications. A “glycopeptide fragment” refers to a peptide fragment resulting from enzymatic or chemical cleavage of a larger polypeptide in which the peptide fragment retains covalently bound carbohydrate. Proteins are glycosylated by well-known enzymatic mechanisms, typically at the side chains of serine or threonine residues (O-linked) or the side chains of asparagine residues (N-linked). N-linked glycosylation sites generally fall into a sequence motif that can be described as N—X—S/T, where X can be any amino acid except proline.

A “sample” means any fluid, tissue, organ or portion thereof, that includes one or more cells, proteins, peptides or peptide fragments. A sample can be a tissue section obtained by biopsy, or cells that are in suspension or are placed in or adapted to tissue culture. A sample can also be a biological fluid specimen such as blood, serum or plasma, cerebrospinal fluid, urine, saliva, seminal plasma, pancreatic juice, breast milk, lung lavage, and the like. A sample can additionally be a cell extract from any species, including eukaryotic cells. A tissue or biological cell sample can be further fractionated, if desired, to a fraction containing particular cell types, portions of cells. Therefore, in certain instances, a sample includes cells in culture, cell supernatants, cell lysates, serum, plasma, biological fluid, and tissue samples.

The term “label” or “labeling” refer to a binding interaction between two or more entities. Where two entities, e.g., molecules or a molecule and a peptide, are bound to each other, they may be directly bound, i.e., bound directly to one another, or they may be indirectly bound, i.e., bound through the use of an intermediate linking moiety or entity. In either case the binding may covalent; e.g., through covalent bonds; or non-covalent, e.g., through ionic bonds, hydrogen bonds, electrostatic interactions, hydrophobic interactions, Van der Waals forces, or a combination thereof. In certain instances, the label is detectable by methods known in the art.

### Methods for Profiling Glycoproteins.

In one aspect of the present disclosure a method for profiling of glycoproteins is provided that includes (a) processing a sample including cells in order to isolate a cytosolic fraction of proteins from the cells and a membrane fraction of proteins from the cells, and (b) performing a mass spectrometry analysis of the proteins in the membrane fraction to obtain a profile of glycoproteins in the membrane fraction, and performing a mass spectrometry analysis of the proteins in the cytosolic fraction to obtain a profile of glycoproteins in the cytosolic fraction.

According to the present method, a population of cells from a sample is processed to obtain a cytosolic fraction of the cells and a membrane fraction of the cells, each of the cellular fractions contain proteins or peptides that are analyzed by mass spectrometry. The mass spectra information obtained from the proteins or peptides is then analyzed or searched against a database comprised of amino acid sequences that encode proteins and/or glycan databases that include the mass spectra of known glycans, glycopeptides, glycoproteins or glycosylation sites (glycosites). As a result of such analysis, the glycoprotein profile of the cytosolic fraction, membrane fraction and whole-cell can be identified for the cells.

In some embodiments, the sample of cells for processing according to the present methods is a sample of eukaryotic cells that may include, but are not limited to, those obtained from animals including humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. In certain embodiments, eukaryotic cells include those obtained from a mammal. In specific embodiments, the sample is a sample of human cells or a sample of mouse cells. In one embodiment, the sample is a sample of human cells. In another embodiment, the sample is a sample of mouse cells.

In certain embodiments, the sample is comprised of adherent cells. In other embodiments, the sample is a suspension of cells. In yet another embodiment, the sample is a soft tissue sample including cells. In other embodiments, the sample is a hard tissue sample including cells. The tissues or cells may be fresh, frozen, dried, cultured, dehydrated, preserved, or maintained by methods known to those of ordinary skill in the art.

As shown in Example 1 and Example 2, the sample of cells can be a sample of adherent human cells. In such instances, the cells are grown in culture and harvested for processing and use in the methods. Generally, the sample of cells should be sufficient in number to generate at least about 400 μg of protein, at least 400 μg of protein, at least 500 μg of protein, at least 600 μg, at least at least 700 μg, at least 800 μg, at least 900 μg, at least 1000 μg, at least 1100 μg, at least 1200 μg, or at least 1300 μg of protein. In a specific embodiment, the sample of cells should generate at least 1200 μg of protein.

In other embodiments, the sample of cells for use in the present methods generates at least 300 μg of membrane protein and at least 700 μg of cytosolic protein. In certain embodiments, the cells generate at least 400 μg of membrane protein and at least 800 μg of cytosolic protein.

In certain embodiments, the sample of cells for use in the present methods includes at least 2.5×10⁶ cells, at least 3.0×10⁶ cells, at least 3.5×10⁶ cells, at least 4.0×10⁶ cells, at least 4.5×10⁶ cells, at least 5.0×10⁶ cells, or more. In a specific embodiment, 2.5×10⁶ cells are processed for use in the present methods.

In other instances, the sample can be a tissue sample containing cells. As shown in Example 1 and Example 3, the tissue sample can be a soft tissue sample including mammalian (e.g., mouse) cells. In embodiments where a tissue sample is used, at least 15 mg of tissue should be obtained. In certain embodiments, at least 25 mg, at least 30 mg, at least 35 mg, at least 40 mg, at least 45 mg or at least 50 mg of tissue is processed. In a specific embodiment, at least 20 mg of tissue is processed. In some embodiments, between 15 mg of tissue and 80 mg of tissue is processed, between 20 mg of tissue and 80 mg of tissue, between 20 mg of tissue and 70 mg of tissue, between 20 mg of tissue and 60 mg of tissue, between 20 mg of tissue and 50 mg of tissue, between 20 mg of tissue and 40 mg of tissue, between 25 mg of tissue and 45 mg of tissue, between 25 mg of tissue and 35 mg of tissue, or between 30 and 40 mg of tissue are used for processing according to the present methods. In one embodiment, between 20 mg and 40 mg of soft tissue is processed. In a specific embodiment, about 30 mg of tissue is processed according to the present methods.

In the present methods, the sample is processed to separate a cytosolic fraction from the cells and a membrane fraction from the cells. The term “cytosolic fraction” or “cytoplasmic fraction” as used herein is a portion of a cell (or collection of cells in a sample) that includes molecules such as, for example, cytoplasm, proteins (including glycoproteins), nucleic acids, peptides, sugars and fats but does not include elements of a cell generally found exclusively in a membrane, such as the plasma membrane or nuclear membrane. In various embodiments, the term cytosolic fraction means a portion of the cell(s) including proteins or peptides or glycoproteins or glycopeptides found in the cytoplasm of cells, but is essentially devoid of proteins or peptides generally found in the membranes of cells. The term “membrane fraction” as used herein is a portion of a cell (or collection of cells in a sample) that includes molecules, such as, for example, lipids, proteins (including glycoproteins), peptides and sugars generally found in a membrane or compartment thereof, such as the plasma membrane or nuclear membrane of a cell. In various embodiments, the term membrane fraction means a portion of the cell(s) including proteins or peptides, including glycoproteins or glycopeptides, found in a membrane of a cell, but is essentially devoid of cytoplasmic proteins or peptides.

According to the inventive methods, a cytosolic fraction is obtained by processing a sample. In various embodiments, processing includes contacting the sample with a permeabilization solution comprising a detergent that permeabilizes the membranes of the cells in the sample to release cytosolic proteins from cells.

In some embodiments, the permeabilization solution includes a first detergent that is mild enough to permeabilize the membranes of cells to permit the release of cytosolic proteins from cellular compartments but does not release transmembrane proteins from membranes. In certain embodiments, the permeabilization solution includes one or more nonionic detergents. In specific embodiments, the nonionic detergent is, for example, 2-[4-(2,4,4-trimethylpentan-2-yl)phenoxy]ethanol (Triton-X 100), octylphenoxypolyethoxyethanol (nonidet P-40, NP-40, IGEPAL CA-630), polysorbate 20 (Tween-20) or Saponin. In certain embodiments, the permeabilization solution includes Triton-X 100. In other embodiments, the permeabilization solution includes octylphenoxypolyethoxyethanol. In yet other embodiments, the permeabilization solution includes polysorbate20 (Polyoxyethylene (20) sorbitan monolaurate). In another embodiment, the permeabilization solution includes Saponin, i.e., triterpene glycoside having the chemical abstract services reference number CAS 8047-15-2. In one instance, the permeabilization solution is the Permeabilization Buffer described in the Mem-PER™ Membrane Protein Extraction Kit (Thermo Scientific™), the entire contents of which is incorporated herein by reference.

The concentration of nonionic detergent in the permeabilization solution can vary depending on, for example, the type or number of nonionic detergents in the permeabilization solution, or additional components of the permeabilization solution. The concentration of nonionic detergent in the permeabilization solution used in accordance with the present methods can be readily determined by one of ordinary skill in the art. For example, in certain embodiments, the permeabilization solution comprises about 0.05%-0.25% weight by volume of nonionic detergent. In another embodiment, the permeabilization solution comprises about 0.10% to 0.20% weight by volume of nonionic detergent. In some embodiments, the permeabilization solution includes about 0.1%-0.15% nonionic detergent. In other embodiments, the permeabilization solution includes 0.15% to 0.20% nonionic detergent. In one embodiment, the permeabilization solution includes 0.10% to 0.20% nonionic detergent.

In some embodiments, the permeabilization solution includes about 0.05%, about 0.10%, about 0.15%, about 0.20% or about 0.25% non-ionic detergent. In specific embodiments, the permeabilization solution includes 0.10% nonionic detergent. In other embodiments, the permeabilization solution includes 0.20% nonionic detergent.

The amount of permeabilization solution used per amount of sample of tissue or amount of cells vary depending on the composition of the permeabilization solution, amount of sample, the type of sample and/or the physical state of, for example, a tissue sample (e.g., hard, soft, dehydrated, fresh, or frozen). Regardless, the amount of permeabilization buffer used in the present methods can be readily determined by one of ordinary skill in the art.

The resulting permeabilized sample is composed of a solution including a mixture or milieu of a cytosolic fraction and a membrane fraction. In certain embodiments, the solution may be mixed by, for example, vortexing or shaking.

This solution is then subjected to centrifugation to obtain a pellet of permeabilized cells, and a supernatant including the cytosolic fraction. In certain embodiments, the solution is centrifuged at about 16,000 g for a period of time sufficient to separate the pellet of permeabilized cells from the supernatant. In some embodiments, the solution is centrifuged at about 16,000 g for at least 10 minutes, at least 8 minutes, at least 6 minutes or at least 5 minutes. In other embodiments, the sample is centrifuged at about 16,000 g for between 5 minutes and 20 minutes, between 10 minutes and 20 minutes, between 10 minutes and 15 minutes, or between 12 minutes and 18 minutes.

In a specific embodiment, the solution is centrifuged at 16,000 g for 15 minutes in order to separate the pellet of permeabilized cells from the supernatant containing the cytosolic fraction.

The supernatant composed of the cytosolic fraction of proteins from the cells is collected by means known by those of ordinary skill in the art, such as, pipetting or aspiration.

The pellet of permeabilized cells is then contacted with a solubilization solution including a second detergent to form a suspension including solubilized membrane proteins from the cells.

Generally, the solubilization solution includes a detergent that is capable of permeabilizing membrane proteins from the permeabilized cells. In certain embodiments, the solubilization solution includes one or more ionic detergents. In specific embodiments, the ionic detergent is, for example, sodium dodecyl sulfate (SDS), sodium deoxycholate, N-lauryl sarcosine or 3-[(3-cholamidopropyl)dimethylammonio]-1-propanesulfonate (CHAPS). In one embodiment, the solubilization solution comprises SDS and sodium deoxycholate. In one embodiment the solubilization solution comprises ionic detergents SDS and sodium deoxycholate as well as a non-ionic detergent such as, for example, octylphenoxypolyethoxyethanol and other components (e.g., sodium chloride (NaCl) and Tris HCl).

In one embodiment, the solubilization solution includes SDS. In another embodiment, solubilization solution includes sodium deoxycholate. In yet another embodiment, the solubilization solution includes N-lauryl sarcosine. In one embodiment, the solubilization solution includes CHAPS. In one instance, the solubilization solution is the Solubilization Buffer described in the Mem-PER™ Membrane Protein Extraction Kit (Thermo Scientific™), the entire contents of which is incorporated herein by reference.

The concentration of ionic detergent in the solubilization solution can vary depending on, for example, the type or number of detergents in the solubilization solution, or additional components of the solubilization solution. The concentration of ionic detergent in the solubilization solution used in accordance with the present methods can be readily determined by one of ordinary skill in the art. For example, in certain embodiments, the solubilization solution comprises about 0.05%-1.5% ionic detergent. In some embodiments, the solubilization solution includes an ionic detergent at a concentration of 0.1% to 1.0% weight by volume of solution. In some embodiments, the solubilization solution includes about 0.1%-0.5% ionic detergent. In other embodiments, the solubilization solution includes 0.1% to 0.2% ionic detergent. In another embodiment, the solubilization solution includes 0.2% to 1.0% ionic detergent. In one embodiment, the solubilization solution includes 0.5% to 1.0% ionic detergent.

In certain embodiments, the solubilization solution includes about 0.1%, about 0.2%, about 0.3%, about 0.5%, about 0.6%, about 0.7%, about 0.8%, about 0.9%, about 1.0% or about 1.2% weight by volume of ionic detergent. In specific embodiments, the solubilization solution includes 0.1% ionic detergent. In other embodiments, the solubilization solution includes 0.2% ionic detergent. In other embodiments, the solubilization solution includes 0.3% ionic detergent. In yet other embodiments, the solubilization solution includes 0.4% ionic detergent. In another embodiment, the solubilization solution includes 0.5% ionic detergent. In yet another embodiment, the solubilization solution includes 0.6% ionic detergent. In other embodiments, the solubilization solution includes 0.7% ionic detergent. In one embodiment, the solubilization solution includes 0.8% ionic detergent. In yet another embodiment, the solubilization solution includes 0.9% ionic detergent. In one embodiment, the solubilization solution includes 1.0% ionic detergent.

For example, in embodiments whereby the solubilization solution comprises SDS, the concentration of SDS can be about 0.1%-1.0% weight by volume. In embodiments whereby the solubilization solution comprises sodium deoxycholate, the concentration of sodium deoxycholate can be about 0.5%-1.0%. In embodiments whereby the solubilization solution comprises N-lauryl sarcosine, the concentration of N-lauryl sarcosine can be about 0.5%-1.0%. In embodiments whereby the solubilization solution comprises CHAPS, the concentration of CHAPS can be about 0.2%-1.0%. In embodiments, whereby the solubilization solution comprises SDS and sodium deoxycholate as well as octylphenoxypolyethoxyethanol, NaCl and Tris HCl, the concentration of SDS in the solubilization solution is about 0.1%, the concentration of sodium deoxycholate in the solubilization solution is 0.5%-1.0%, the concentration of NaCl is about 100-175 mM, and the concentration of Tris HCl is about 25-75 mM at neutral pH (e.g., pH 8).

The amount of solubilization solution used per weight of tissue or amount of cells vary depending on the amount of sample, the type of sample and/or the physical state of, for example, a tissue sample (e.g., hard, soft, dehydrated, fresh, or frozen). Regardless, the amount of solubilization buffer used in the present methods can be readily determined by one of ordinary skill in the art.

In certain embodiments, the suspension of solubilized membrane proteins may be mixed by, for example, vortexing or shaking.

The suspension of solubilized membrane proteins is then subjected to centrifugation to obtain a pellet and a supernatant including the membrane fraction. In certain embodiments, the suspension of solubilized membrane proteins is centrifuged at about 16,000 g for a period of time sufficient to separate the pellet from the supernatant. In some embodiments, the suspension is centrifuged at about 16,000 g for at least 10 minutes, at least 8 minutes, at least 6 minutes or at least 5 minutes. In other embodiments, the suspension is centrifuged at about 16,000 g for between 5 minutes and 20 minutes, between 10 minutes and 20 minutes, between 10 minutes and 15 minutes, or between 12 minutes and 18 minutes.

In a specific embodiment, the suspension of solubilized membrane proteins is centrifuged at 16,000 g for 15 minutes in order to separate the pellet from the supernatant containing the membrane fraction.

The supernatant composed of the membrane fraction of proteins from the cells is collected, by means known by those of ordinary skill in the art, such as, pipetting or aspiration.

### Mass Spectrometry Analysis of Cytosolic Fractions and Membrane Fractions.

To obtain a profile of glycoproteins according to the present methods (i.e., profile of glycoproteins from the membrane fraction and profile of glycoproteins from the cytosolic fraction) the collected membrane fraction(s) and cytosolic fraction(s) are analyzed by mass spectrometry to obtain mass spectra.

In some embodiments, the profile of glycoproteins identified by mass spectrometry analysis of the membrane fraction of proteins and/or cytosolic fraction of proteins is obtained by a process that includes digesting proteins in the membrane fraction to obtain a sample of peptide fragments from the membrane fraction and/or digesting the proteins in the cytosolic fraction to obtain a sample of peptide fragments from the cytosolic fraction.

In some embodiments, mass spectra information is obtained from glycoproteins or glycopeptide fragments which are generated from the proteins within a membrane fraction or a cytosolic fraction. For example, the glycoproteins in a fraction can be fragmented, such as, by one or more proteases, and/or a chemical protein cleavage reagent, such as cyanogen bromide. A non-comprehensive list of known proteases for the fragmentation of proteins includes: trypsin (cleaving at argentine or lysine, unless followed by Pro), chymotrypsin (cleaves after Phe, Trp, or Tyr, unless followed by Pro), elastase (cleaves after Ala, Gly, Ser, or Val, unless followed by Pro), pepsin (cleaves after Phe or Leu), and thermolysin (cleaves before Ile, Met, Phe, Trp, Tyr, or Val, unless preceded by Pro). A more comprehensive listing of proteases that can be used to digest proteins to fragments is provided in Tables 11.1.1 and 11.1.3 of Riviere and Tempst. Curr Protoc Protein Sci. Vol. 0 pp. 11.1.1-11.1.19 (1995) the entire contents of which are herein incorporated by reference.

Proteins may be digested to smaller fragments that are amenable to mass spectrometry by treatment with particular chemical protein cleavage reagents rather than proteolytic enzymes. See for example chapter 3 of G. Allen, Sequencing of Proteins and Peptides, Laboratory Techniques in Biochemistry and Molecular Biology, Vol. 9. Elsevier 1989. Such chemical protein cleavage reagents include, without limitation, cyanogen bromide, BNPS-skatole, o-iodosobenzoic acid, dilute acid (e.g., dilute HC), and so forth. For example, proteins can be cleaved at Met residues with cyanogen bromide, at Cys residues after cyanylation, after Trp residues with BNPS-skatole or o-iodosobenzoic acid, etc. Protein fragments can also be generated by exposure to dilute acid, e.g., HCl. An example of the use of partial acid hydrolysis to determine protein sequences by mass spectrometry is given by Zhong et al. (Zhong H, et al., J. Am. Soc. Mass Spectrom. 16(4):471-81, 2005, incorporated by reference in its entirety). Zhong et al., supra used microwave-assisted acid hydrolysis with 25% trifluoroacetic acid in water to fragment bacteriorhodopsin for sequencing by mass spectrometry. See also Wang N, and Li L., J. Am. Soc. Mass. Spectrom. 21(9):1573-87, 2010, the entire contents of which is incorporated herein by reference.

Proteins can be fragmented by treatment with one protease, by treatment with more than one protease in combination, by treatment with a chemical cleavage reagent, by treatment with more than one chemical cleavage reagent in combination, or by treatment with a combination of proteases and chemical cleavage reagents. The reactions may occur at elevated temperatures or elevated pressures. See for example Lopez-Ferrer D, et al., J. Proteome. Res. 7(8):3276-81, 2008 (incorporated by reference in its entirety). The fragmentation can be allowed to go to completion so the protein is cleaved at all bonds that the digestion reagent is capable of cleaving; or the digest conditions can be adjusted so that fragmentation does not go to completion deliberately, to produce larger fragments that may be particularly helpful in deciphering antibody variable region sequences; or digest conditions may be adjusted so the protein is partially digested into domains, e.g., as is done with E. coli DNA polymerase I to make Klenow fragment. The conditions that may be varied to modulate digestion level include duration, temperature, pressure, pH, absence or presence of protein denaturing reagent, the specific protein denaturant (e.g., urea, guanidine HCl, detergent, acid-cleavable detergent, methanol, acetonitrile, other organic solvents), the concentration of denaturant, the amount or concentration of cleavage reagent or its weight ratio relative to the protein to be digested, among other things.

In some embodiments, the reagent (i.e., the protease or the chemical protein cleavage reagents) used to cleave the proteins is a completely non-specific reagent. Using such a reagent, no constraints are made may be made at the N-terminus of the peptide, the C-terminus of the peptide, or both of the N- and C-termini. For example, a partially proteolyzed sequence that is constrained to have a tryptic cleavage site at one end of the peptide sequence or the other, but not both, may be used in the various methods described herein.

In certain embodiments, the digestion is carried out by Filter Assisted Sample Preparation (FASP) as described in Example 1.

In various embodiments, the protein fragments or proteins obtained from the cytosolic fraction(s) and membrane fraction(s) can then be fractionated in order to separate non-glycosylated proteins from glycoproteins in each fraction, and thus “enrich” the samples to be analyzed by mass spectrometry proteins from each of the cytosolic and membrane fractions for glycoproteins or glycopeptides fragments.

In certain instances, the peptide fragments from the cytosolic fraction or the membrane fraction of proteins are enriched by separating non-glycosylated peptides from glycopeptides through hydrophilic interaction liquid chromatography (HILIC), lectin affinity chromatography, or hydrazide capture. In a specific embodiment, the sample of peptide fragments from the cytosolic fraction is enriched by HILIC.

As shown in Example 1, in an exemplary embodiment, the peptide fragments from the membrane fraction(s) and the cytosolic fraction(s) of proteins are enriched by HILIC. Here, peptide fragments from cytosolic and membrane fractions were separated individually on a amide column and each separated subset (fraction) of proteins from the membrane fraction and cytosolic fraction were collected. Subsets containing glycosylated peptide fragments were then isolated for further use.

In some embodiments, the present methods include releasing the glycans from the enriched samples of glycoproteins or glycopeptide fragments. In one embodiment, glycans are released from enriched sample of glycopeptides fragments from the cytosolic fraction of proteins by contacting the sample with a glycosidase, such as an amidase. In another embodiment, glycans are released from enriched sample of glycopeptides fragments from the membrane fraction of proteins by contacting the sample with a glycosidase, such as an amidase.

In a specific embodiment, N-linked glycans are released from glycopeptide fragments or glycoprotein by Peptide-N-Glycosidase F (PNGase F).

The methods of the present disclosure can be used to identify and/or quantify the amount or type of a glycoprotein present in a sample or fraction thereof. A particularly useful method for identifying and quantifying a glycoprotein or glycopeptide fragment is mass spectrometry (MS). The methods of the disclosure can be used to identify a glycoprotein or glycopeptide fragment qualitatively, for example, using MS analysis. For example, a glycopeptide fragment can be labeled using a detectable marker to facilitate quantitative analysis by, for example, liquid chromatography-mass spectrometry (LCMS).

In embodiments, where quantitative analysis of the glycoprotein or glycopeptide fragments is desired, the glycoproteins or glycopeptide fragments in a cytosolic fraction and membrane fraction is labeled with a detectable marker. For example, a detectable marker suitable for use in the present methods is a chemical moiety having suitable chemical properties for incorporation of an isotope, allowing the generation of chemically identical reagents of different mass which can be used to (differentially) identify a polypeptide in two fractions.

Isotopes have traditionally been incorporated into peptides and proteins by numerous chemical, enzymatic, and metabolic labeling methods. Enzymatic methods for isotope labeling generally add ¹⁸O isotopes to peptide carboxyl termini through tryptic digestion in ¹⁸O-labeled water. Stable isotopes can be metabolically incorporated into proteins in cell culture (stable isotope cell culture, SILAC). SILAC methods use metabolic incorporation into proteins of heavy isotope-labeled amino acids or non-heavy isotope-labeled, i.e., unlabeled or light, amino acids. Heavy isotopes that can be used are stable isotopes such as, but not limited to, ¹³O, ¹⁵N, ⁷⁴Se, ⁷⁶Se, ⁷⁷Se, ⁷⁸Se, ⁸²Se, ¹⁸O, and ²H. An example of the SILAC technique used for metabolic incorporation of isotopes uses Escherichia coli (E. coli) cultured with media supplemented with heavy isotope-labeled amino acids to express isotope-labeled proteins or concatenated polypeptides (QConCat).

Another common labeling method uses chemically synthesized isotope-labeled peptides for absolute quantitation, i.e., AQUA method. The AQUA method introduces known quantities of isotope-labeled peptides into biological samples to be analyzed, permitting the relative quantification of unlabeled peptides. Absolute quantitation can be accomplished by classic isotope dilution measurements, where stable isotope-labeled peptides are used to generate a standard curve.

For example, an isobaric tag or isotope tag (i.e., a detectable marker) has an appropriate composition to allow incorporation of a stable isotope at one or more atoms. A particularly useful stable isotope pair is hydrogen and deuterium, which can be readily, distinguished using mass spectrometry as light and heavy forms, respectively. Any of a number of isotopic atoms can be incorporated into the isotope tag so long as the heavy and light forms can be distinguished using mass spectrometry, for example, ¹³C, ¹⁵N, ¹⁷O, ¹⁸O or ³⁴S. Other exemplary isotope tags will also be known to those of ordinary skill in the art, such as the 4,7,10-trioxa-1,13-tridecanediamine based linker and its related deuterated form, 2,2′,3,3′,11,11′,12,12′-octadeutero-4,7,10-trioxa-1,13-tridecanediamine, described by Gygi et al. Nature Biotechnol. 17:994-999 (1999) the entire contents of which is hereby incorporated by reference.

Alternatively, peptides in a sample or fraction can be labeled using isotopic or isobaric chemical tags, e.g., isotope dimethylation, iCAT, iTRAQ or TMT reagents to create internal reference peptide standards for relative quantitation. These methods conjugate and/or covalently attach chemical tags to peptides and/or proteins.

Both peptide and protein isotope labeling are applicable for relative and absolute MS quantitation.

As shown in Example 1, proteins and/or peptide fragments from the cytosolic fraction(s) and membrane fraction(s) of a sample were labeled using Tandem Mass Tag™ (TMT) system (Thermo Scientific™). The exemplary detectable label utilized (Tandem Mass Tag) is an isobaric detectable marker that covalently labels primary amines (—NH2 groups) or lysine residues of peptides. The exemplary isobaric detectable marker contains heavy isotopes, which are detectable in mass specification for sample identification and quantitation of peptides.

The inventive method of profiling glycoproteins includes performing a mass spectrometry analysis of the peptide fragments obtained from each of the cytosolic fractions and membrane fractions of a sample in order to obtain the profile of glycoproteins in the membrane fraction and/or the profile of glycoproteins in the membrane fraction.

Mass spectra information can be obtained by mass spectrometry analysis of collected fractions or peptide fragments generated therefrom. A mass spectrometer is an instrument capable of measuring the mass-to-charge (m/z) ratio of individual ionized molecules, allowing researchers to identify unknown compounds, to quantify known compounds, and to elucidate the structure and chemical properties of molecules. In some embodiments, one begins mass spectrometry analysis by isolating and loading a sample onto the instrument. Once loaded, the sample is vaporized and then ionized. Subsequently, the ions are separated according to their mass-to-charge ratio via exposure to a magnetic field. In some embodiments, a sector instrument is used, and the ions are quantified according to the magnitude of the deflection of the ion's trajectory as it passes through the instrument's electromagnetic field, which is directly correlated to the ions mass-to-charge ratio. In other embodiments, ion mass-to-charge ratios are measured as the ions pass through quadrupoles, or based on their motion in three dimensional or linear ion traps or Orbitrap, or in the magnetic field of a Fourier transform ion cyclotron resonance mass spectrometer. The instrument records the relative abundance of each ion, which is used to determine the chemical, molecular and/or isotopic composition of the original sample. In some embodiments, a time-of-flight instrument is used, and an electric field is utilized to accelerate ions through the same potential, and measures the time it takes each ion to reach the detector. This approach depends on the charge of each ion being uniform so that the kinetic energy of each ion will be identical. The only variable influencing velocity in this scenario is mass, with lighter ions traveling at larger velocities and reaching the detector faster consequently. The resultant data is represented in a mass spectrum or a histogram, intensity vs. mass-to-charge ratio, with peaks representing ionized proteins or peptide fragments.

After passage of the mass spectrometry analysis is performed, numerous the mass spectra for a sample or fraction thereof is generated. However, given the potentially large number of different glycoproteins, glycans, glycosites and/or glycopeptides within a fraction or sample, each with a different amino acid sequence, that are analyzed with the mass spectrometer, the actual glycoproteins, glycan composition, and glycopeptides may be difficult to identify. Therefore, in various embodiments, the inventive methods include comparing or searching the actual mass spectral data through a database or search engine of proteins/peptides such as the UNIPROT database and a glycan and/or glycoprotein search engine (e.g., Byonic™ or SimGlycan) to be correlated with the predicted mass spectra of the protein sequence to obtain the amino acid sequence of the glycoprotein or fragment thereof.

More specifically, by correlating the predicted mass spectra information from the database or search engine with the observed mass spectra information from the actual glycoproteins or glycopeptides fragments generated above, those glycoproteins, glycans, glycopeptides or glycosites in the database can be selected that correspond to actual mass spectra identified.

By “correlating” it is meant that the observed mass spectra information derived from the peptide fragments or glycoproteins in a cytosolic and/or membrane fraction prepared according to the present methods and the predicted mass spectra information derived from a database are cross-referenced and compared against each other, such that peptide fragments or glycoproteins can be identified or selected from the database that correspond to peptide fragments or glycoproteins in a cytosolic and/or membrane fraction.

In specific embodiments, the correlating process involves comparing the recorded mass spectra from a cytosolic or membrane fraction with the predicted spectra information to identify matches. For example, each of the recorded spectra can be searched against the collection of predicted mass spectra derived from a database, with each predicted spectrum being identifiably associated with a peptide sequence or glycan from the database. Once a match is found, i.e., an recorded mass spectrum is matched to a predicted mass spectrum, because each predicted mass spectrum is identifiably associated with a peptide sequence in the database, the recorded mass spectrum is said to have found its matching peptide sequence—such match also referred to herein as “peptide spectrum match” or “PSM”. Because of the large number of spectra to be searched and matched, this search and matching process can be performed by computer-executed functions and softwares, such as the Uniprot human proteome database, the Uniprot mouse proteome database, a Byonic™ human glycan database and/or a Byonic™ mammalian glycan database in order to identify the glycopeptides, PSM, glycoproteins, glycan composition and/or glycosylation sites in each fraction.

In some embodiments, the glycoprotein profile identifies a listing of glycoproteins. In certain embodiments, the glycoprotein profile identifies one or more of the following characteristics: a glycosylation site, glycopeptide quantity in a fraction, glycan composition, or abundance of the glycoproteins.

In further embodiments, the method of profiling glycoproteins includes obtaining the glycoproteomic profile of a cytosolic fraction of proteins and/or a membrane fraction of proteins by searching the mass spectra data from the cytosolic fraction of proteins and/or a membrane fraction of proteins against a proteome database. In some embodiments, the proteome database is the Uniprot human proteome database or the Uniprot mouse proteome database.

In one embodiment, the sample of cells includes human cells and the mass spectra data from the cytosolic fraction of proteins and/or a membrane fraction of proteins is searched against the Uniprot human proteome database.

In another embodiment, the sample of cells includes murine cells and the mass spectra data from the cytosolic fraction of proteins and/or a membrane fraction of proteins is searched against the Uniprot mouse proteome database.

In various embodiments, profiling glycoproteins includes obtaining the glycoproteomic profile of a cytosolic fraction of proteins and/or a membrane fraction of proteins by searching the recorded mass spectra data from the cytosolic fraction of proteins and/or a membrane fraction of proteins against a proteome database and a glycan database. In certain embodiments, the sample of cells includes human cells and the mass spectra data from the cytosolic fraction of proteins and/or a membrane fraction of proteins is searched against the Uniprot human proteome database and a human glycan database, such as the Byonic™ human glycan database in order to identify the glycopeptides, PSM, glycoproteins, glycan composition and glycosylation sites in each fraction. See Example 2.

In another embodiment, the sample of cells includes murine cells and the mass spectra data from the cytosolic fraction of proteins and/or a membrane fraction of proteins is searched against the Uniprot mouse proteome database and a murine glycan database such as, for example, the Byonic™ mammalian glycan database in order to identify the glycopeptides, PSM, glycoproteins, glycan composition and/or glycosylation sites in each fraction. See Example 3.

In yet another embodiment, the profile of glycoproteins in the cytoplasmic fraction and the profile of glycoproteins in the membrane fraction of cells obtained by the present methods are compared in order to obtain the unique number of glycosylation sites, glycopeptides, glycans, and/or glycoproteins in each fraction or in the whole-cell.

By “unique number of”, it is meant the number of distinct glycosylation sites, glycopeptides, glycans, and/or glycoproteins observed in a fraction or sample.

### Method for Detecting Protein Variation Between Samples or Preparations Thereof

The present disclosure also recognizes that the present methods can be used to determine the variability in proteins across samples or across preparations of samples. For example, the inventors have shown that the present methods consistently isolate glycoproteins from the cytosol and membranes of cells in a single process, and identified a use for such method to, for example, determine whether or not a variation in the protein production, protein location or post-translational modification of proteins exists across samples or preparations thereof.

Therefore, in another aspect of the present disclosure a method for detecting protein variation between samples or preparations of samples is provided. In one embodiment, the method for detecting protein variation includes (a) processing a first sample including cells in order to isolate a cytosolic fraction of proteins from the cells and a membrane fraction of proteins from the cells of the first sample, and (b) processing a second sample composed of cells in order to isolate a cytosolic fraction of proteins from the cells and a membrane fraction of proteins from the cells of the second sample, and (c) digesting the proteins in the cytosolic and membrane fractions in the first sample in order to obtain peptide fragments from the cytosolic fraction and obtain peptide fragments the membrane fraction from the cells of the first sample, and (d) digesting the proteins in the cytosolic and membrane fractions in the second sample in order to obtain peptide fragments from the cytosolic fraction and obtain peptide fragments from the membrane fraction from the cells of the second sample, and (e) labeling the peptide fragments in the cytosolic fraction from the first sample (i.e., with a detectable marker) and labeling the peptide fragments in the cytosolic fraction from the second sample, and mixing the labeled cytosolic fractions to obtain a mixture of labeled cytosolic peptide fragments from the first and second samples (or preparations thereof, and (f) labeling the peptide fragments in the membrane fraction from the first sample (i.e., with a detectable marker) and labeling the peptide fragments in the membrane fraction of cells from the second sample, mixing the labeled membrane fractions to obtain a mixture of labeled membrane peptide fragments from the first and second samples, and (g) detecting the cytosolic peptide fragments in the mixture of labeled cytosolic peptide fragments; and detecting the membrane peptide fragments in the mixture of labeled membrane peptide fragments, thereby determining whether or not any variation in the total amount of cytosolic proteins and/or membrane proteins exists between the first sample and the second sample.

The cytosolic and membrane fractions are procured as stated herein. Accordingly, the inventive methods, a cytosolic fraction is obtained by processing a sample. In various embodiments, processing includes contacting the sample with a permeabilization solution comprising a first detergent that permeabilizes the membranes of cells in the sample to release cytosolic proteins from the cells.

In various embodiments, processing includes contacting the sample with a permeabilization solution comprising a detergent that permeabilizes the membranes of the cells in the sample to release cytosolic proteins from cells. In some embodiments, the permeabilization solution includes a first detergent that is mild enough to permeabilize the membranes of cells to permit the release of cytosolic proteins from cellular compartments but does not release transmembrane proteins from membranes. In certain embodiments, the permeabilization solution includes one or more nonionic detergents. In specific embodiments, the nonionic detergent is, for example, 2-[4-(2,4,4-trimethylpentan-2-yl)phenoxy]ethanol (Triton-X 100), octylphenoxypolyethoxyethanol (nonidet P-40, NP-40, IGEPAL CA-630), polysorbate 20 (Tween-20) or Saponin. In certain embodiments, the permeabilization solution includes Triton-X 100. In other embodiments, the permeabilization solution includes octylphenoxypolyethoxyethanol. In yet other embodiments, the permeabilization solution includes polysorbate20 (Polyoxyethylene (20) sorbitan monolaurate). In another embodiment, the permeabilization solution includes Saponin, i.e., triterpene glycoside having the chemical abstract services reference number CAS 8047-15-2. In one instance, the permeabilization solution is the Permeabilization Buffer described in the Mem-PER™ Membrane Protein Extraction Kit (Thermo Scientific™), the entire contents of which is incorporated herein by reference.

The concentration of nonionic detergent in the permeabilization solution can vary depending on, for example, the type or number of nonionic detergents in the permeabilization solution, or additional components of the permeabilization solution. The concentration of nonionic detergent in the permeabilization solution used in accordance with the present methods can be readily determined by one of ordinary skill in the art. For example, in certain embodiments, the permeabilization solution comprises about 0.05%-0.25% weight by volume of nonionic detergent. In another embodiment, the permeabilization solution comprises about 0.10% to 0.20% weight by volume of nonionic detergent. In some embodiments, the permeabilization solution includes about 0.1%-0.15% nonionic detergent. In other embodiments, the permeabilization solution includes 0.15% to 0.20% nonionic detergent. In one embodiment, the permeabilization solution includes 0.10% to 0.20% nonionic detergent.

In some embodiments, the permeabilization solution includes about 0.05%, about 0.10%, about 0.15%, about 0.20% or about 0.25% non-ionic detergent. In specific embodiments, the permeabilization solution includes 0.10% nonionic detergent. In other embodiments, the permeabilization solution includes 0.20% nonionic detergent.

The amount of permeabilization solution used per weight of tissue or amount of cells vary depending on the amount of sample, the type of sample and/or the physical state of, for example, a tissue sample (e.g., hard, soft, dehydrated, fresh, or frozen). Regardless, the amount of permeabilization buffer used in the present methods can be readily determined by one of ordinary skill in the art.

The resulting permeabilized sample(s) include a solution having a mixture or milieu of a cytosolic fraction and a membrane fraction. In certain embodiments, the solution may be mixed by, for example, vortexing or shaking.

This solution is then subjected to centrifugation to obtain a pellet of permeabilized cells, and a supernatant including the cytosolic fraction. In certain embodiments, the solution is centrifuged at about 16,000 g for a period of time sufficient to separate the pellet of permeabilized cells from the supernatant. In some embodiments, the solution is centrifuged at about 16,000 g for at least 10 minutes, at least 8 minutes, at least 6 minutes or at least 5 minutes. In other embodiments, the sample is centrifuged at about 16,000 g for between 5 minutes and 20 minutes, between 10 minutes and 20 minutes, between 10 minutes and 15 minutes, or between 12 minutes and 18 minutes.

In a specific embodiment, the solution is centrifuged at 16,000 g for 15 minutes in order to separate the pellet(s) of permeabilized cells from the supernatant containing the cytosolic fraction.

The supernatant composed of the cytosolic fraction of proteins from the cells is collected by means known by those of ordinary skill in the art, such as, pipetting or aspiration.

The pellet(s) of permeabilized cells is then contacted with a solubilization solution including a second detergent to form a suspension including solubilized membrane proteins from the cells. Generally, the solubilization solution includes a detergent that is capable of solubilizing membrane proteins from the permeabilized cells. In certain embodiments, the solubilization solution includes one or more ionic detergents. In specific embodiments, the ionic detergent is, for example, sodium dodecyl sulfate (SDS), sodium deoxycholate, N-lauryl sarcosine or 3-[(3-cholamidopropyl)dimethylammonio]-1-propanesulfonate (CHAPS). In one embodiment, the solubilization solution comprises SDS and sodium deoxycholate. In one embodiment the solubilization solution comprises ionic detergents SDS and sodium deoxycholate as well as a non-ionic detergent such as, for example, octylphenoxypolyethoxyethanol and other components (e.g., sodium chloride (NaCl) and Tris HCl).

In one embodiment, the solubilization solution includes SDS. In another embodiment, solubilization solution includes sodium deoxycholate. In yet another embodiment, the solubilization solution includes N-lauryl sarcosine. In one embodiment, the solubilization solution includes CHAPS. In one instance, the solubilization solution is the Solubilization Buffer described in the Mem-PER™ Membrane Protein Extraction Kit (Thermo Scientific™), the entire contents of which is incorporated herein by reference.

The concentration of ionic detergent in the solubilization solution can vary depending on, for example, the type or number of detergents in the solubilization solution, or additional components of the solubilization solution. The concentration of ionic detergent in the solubilization solution used in accordance with the present methods can be readily determined by one of ordinary skill in the art. For example, in certain embodiments, the solubilization solution comprises about 0.05%-1.5% ionic detergent. In some embodiments, the solubilization solution includes an ionic detergent at a concentration of 0.1% to 1.0% weight by volume of solution. In some embodiments, the solubilization solution includes about 0.1%-0.5% ionic detergent. In other embodiments, the solubilization solution includes 0.1% to 0.2% ionic detergent. In another embodiment, the solubilization solution includes 0.2% to 1.0% ionic detergent. In one embodiment, the solubilization solution includes 0.5% to 1.0% ionic detergent.

In certain embodiments, the solubilization solution includes about 0.1%, about 0.2%, about 0.3%, about 0.5%, about 0.60%, about 0.7%, about 0.8%, about 0.9%, about 1.0% or about 1.2% weight by volume of ionic detergent. In specific embodiments, the solubilization solution includes 0.1% ionic detergent. In other embodiments, the solubilization solution includes 0.2% ionic detergent. In other embodiments, the solubilization solution includes 0.3% ionic detergent. In yet other embodiments, the solubilization solution includes 0.4% ionic detergent. In another embodiment, the solubilization solution includes 0.5% ionic detergent. In yet another embodiment, the solubilization solution includes 0.6% ionic detergent. In other embodiments, the solubilization solution includes 0.7% ionic detergent. In one embodiment, the solubilization solution includes 0.8% ionic detergent. In yet another embodiment, the solubilization solution includes 0.9% ionic detergent. In one embodiment, the solubilization solution includes 1.0% ionic detergent.

For example, in embodiments whereby the solubilization solution comprises SDS, the concentration of SDS can be about 0.1%-1.0% weight by volume. In embodiments whereby the solubilization solution comprises sodium deoxycholate, the concentration of sodium deoxycholate can be about 0.5%-1.0%. In embodiments whereby the solubilization solution comprises N-lauryl sarcosine, the concentration of N-lauryl sarcosine can be about 0.5%-1.0%. In embodiments whereby the solubilization solution comprises CHAPS, the concentration of CHAPS can be about 0.2%-1.0%. In embodiments, whereby the solubilization solution comprises SDS and sodium deoxycholate as well as octylphenoxypolyethoxyethanol, NaCl and Tris HCl, the concentration of SDS in the solubilization solution is about 0.1%, the concentration of sodium deoxycholate in the solubilization solution is 0.5%-1.0%, the concentration of NaCl is about 100-175 mM, and the concentration of Tris HCl is about 25-75 mM at neutral pH (e.g., pH 8).

The amount of solubilization solution used per weight of tissue or amount of cells vary depending on the amount of sample, the type of sample and/or the physical state of, for example, a tissue sample (e.g., hard, soft, dehydrated, fresh, or frozen). Regardless, the amount of solubilization buffer used in the present methods can be readily determined by one of ordinary skill in the art.

In certain embodiments, the suspension of solubilized membrane proteins may be mixed by, for example, vortexing or shaking.

The suspension of solubilized membrane proteins is then subjected to centrifugation to obtain a pellet and a supernatant including the membrane fraction. In certain embodiments, the suspension of solubilized membrane proteins is centrifuged at about 16,000 g for a period of time sufficient to separate the pellet from the supernatant. In some embodiments, the suspension is centrifuged at about 16,000 g for at least 10 minutes, at least 8 minutes, at least 6 minutes or at least 5 minutes. In other embodiments, the suspension is centrifuged at about 16,000 g for between 5 minutes and 20 minutes, between 10 minutes and 20 minutes, between 10 minutes and 15 minutes, or between 12 minutes and 18 minutes.

Ina specific embodiment, the suspension of solubilized membrane proteins is centrifuged at 16,000 g for 15 minutes in order to separate the pellet from the supernatant containing the membrane fraction.

The supernatant composed of the membrane fraction of proteins from the cells is collected, by means known by those of ordinary skill in the art, such as, pipetting or aspiration.

The method for detecting protein variation between samples or preparations of samples includes labeling each fraction (such as, with a detectable marker). In some instances, labeling includes contacting the sample or preparation thereof with a detectable marker. For example, each of the cytosolic fractions obtained from the first and second sample of cells can be labeled with a detectable marker that are the same or different. In one instance, the detectable marker for each of the cytosolic fractions obtained from the first and second sample of cells are different. In some instances, the detectable marker for each of the cytosolic fractions obtained from the first and second sample of cells are the same. In certain instances, the detectable marker for each of the membrane fractions obtained from the first and second sample of cells are different. In some instances, the detectable marker for each of the membrane fractions obtained from the first and second sample of cells are the same. In some embodiments, the detectable markers used to label peptide fragments in each cytosolic fraction are different from one another, and the same detectable markers are used to label peptide fragments in the membrane fraction of the first and second sample of cells. In specific embodiments, the detectable markers are used to label peptide fragments in each cytosolic fraction are the same as the detectable markers used to label peptide fragments in each membrane fraction.

In some embodiments, labeling includes contacting peptide fragments or proteins with isobaric detectable markers that covalently label primary amines (—NH2 groups) and/or lysine residues. In certain embodiments, the isobaric detectable marker contains heavy isotopes, which are detectable in mass spectrometry for sample identification and quantitation of peptides. In a specific embodiment, the proteins or peptides are labeled with isobaric detectable markers as described in the Thermo Scientific™ Tandem Mass Tag (TMT) system (Thermo Scientific™), the entire contents of which is incorporated herein by reference.

As indicated above, in various embodiments, the labeled cytosolic fractions of digested peptides from a sample or sample preparation were combined. For example, TMT labeled membrane fractions of digested peptides from human adherent cell samples were mixed to provide a mixture of labeled membrane peptide fragments from the first and second samples or preparations thereof. Additionally, TMT labeled cytosolic fractions of digested peptides from human adherent cell samples were mixed to provide a mixture of labeled cytosolic peptide fragments from the first and second samples or preparations thereof. See Example 4.

In another embodiment, TMT labeled fractions of digested proteins from soft tissue obtained from mouse liver tissue samples or preparations thereof were combined. As shown in Example 5, TMT labeled membrane fractions of digested peptides from soft tissue samples obtained from mouse liver were mixed to provide a mixture of labeled membrane peptide fragments from the first and second samples or preparations thereof. Additionally, TMT labeled cytosolic fractions of digested peptides from soft tissue samples obtained from mouse liver were mixed to provide a mixture of labeled cytosolic peptide fragments from the first and second samples or preparations thereof.

In other embodiments, the detectable markers are colormetric markers, such as those that identify the peptide bonds and the presence of amino acids (i.e., cysteine, cystine, tryptophan and tyrosine) in the presence of bicinchoninic acid (BCA). In such embodiments, the labeled proteins from each fraction of each sample are detected on visible light spectrophotometer at 562 nm. BCA assays for the detection and quantitation of total protein in a sample are well known to those of ordinary skill in the art. One such BCA assay is The BCA™ Protein Assay as set forth in the BCA™ Protein Assay Kit (Pierce™), the entire contents of which is hereby incorporated by reference.

In various embodiments, the inventive methods include performing a mass spectrometry analysis of a mixture of labeled cytosolic peptides to obtain a profile of glycoproteins in the cytosolic fractions of the first and second samples, and performing a mass spectrometry analysis of a mixture of labeled membrane peptides to obtain a profile of glycoproteins in the membrane fractions of the first and second samples. In certain embodiments, mass spectrometry is performed on the mixture of labeled cytosolic to obtain the profile of glycoproteins in the cytosolic fractions of the first sample and the profile of glycoproteins in the cytosolic fraction of the second sample, wherein each of said profiles comprise a listing of glycoproteins, optionally with one or more of glycosylation sites, glycopeptides, glycan composition, and abundance of the glycoproteins.

In other embodiments, the present methods include separating non-glycosylated peptide fragments from each of the mixtures of cytosolic peptide fragments to obtain a collection of cytosolic peptide fragments from the first sample and second sample enriched in glycosylated peptide fragments. In certain embodiments, non-glycosylated peptide fragments are separated from each of the mixtures of membrane peptide fragments to obtain a collection of membrane peptide fragments from the first sample and second sample enriched in glycosylated peptide fragments.

In some instances, the samples of peptide fragments from the mixture of cytosolic peptide fragments and/or the mixture of membrane peptide fragments are enriched by removing non-glycosylated peptides through ion-pairing hydrophilic interaction liquid chromatography (HILIC), lectin affinity chromatography, or hydrazide capture. In a specific embodiment, the mixture of cytosolic peptide fragments is enriched by ion-pairing HILIC. In another embodiment, the mixture of membrane peptide fragments of proteins is enriched by ion-pairing HILIC.

In some embodiments, the methods include releasing the glycans from the enriched samples of glycoproteins or peptide fragments. In one embodiment, glycans are released from an enriched sample of peptides fragments from the mixture of cytosolic peptide fragments by contacting the mixture with a glycosidase, such as an amidase. In another embodiment, glycans are released from an enriched mixture of membrane peptide fragments by contacting the mixture with a glycosidase, such as an amidase.

In certain embodiments, the inventive method can also be adapted to obtain a glycoprotein profile by performing a mass spectrometry analysis of the peptide fragments obtained from each of the mixed cytosolic fractions and membrane fractions.

Mass spectra information can be obtained by mass spectrometry analysis of collected fractions or peptide fragments generated therefrom as stated above.

### EXAMPLES

### Example 1. Materials and Methods

Sample processing. Protein extraction from human adherent cell sample. Human K562 bone marrow cells (ATCC® CCL-243™), were grown to confluence in cell culture medium according to manufacturers protocol. 2.5×10⁶ K562 cells were harvested and resuspended in 5 mL 1× Phosphate saline buffer (PBS) and centrifuged at 300×g for 5 minutes. The resulting cell pellet was then washed in 2 mL of Cell Wash Solution (Mem-PER™ Plus Membrane Protein Extraction Kit, Thermo Scientific™). The supernatant was discarded and the cell pellet was resuspended in 1.5 mL of Cell Wash Solution. The resulting mixture was transferred to a 2 mL centrifuge tube and centrifuged at 300×g for 5 minutes. The supernatant was discarded and 0.4 mL of Permeabilization Buffer (Mem-PER™ Plus Membrane Protein Extraction Kit, Thermo Scientific™) was added, the cell pellet and Permeabilization Buffer was vortexed to generate a homogeneous suspension. The suspension was then incubated for 10 minutes at 4° C. with constant mixing to release cytosolic proteins from the permeabilized cells. The homogenous suspension of permeabilized cells was then centrifuged for 15 minutes at 16,000×g. The supernatant containing the cytosolic fraction of proteins from the permeabilized cells were collected and transferred to a new receptacle.

To obtain the membrane fraction of proteins from the K562 cell sample, the pellet of permeabilized cells was resuspended in 0.25 mL of Solubilization Buffer (Mem-PER™ Plus Membrane Protein Extraction Kit, Thermo Scientific™) and mixed by pipetting. The suspension was then incubated for 30 minutes at 4° C. with constant mixing to release the solubilized membrane proteins into solution. The suspension was then centrifuged for 15 minutes at 16,000×g and the supernatant containing the membrane fraction of proteins from the cells were collected and transferred to a new receptacle.

Protein extraction from a murine liver (soft) tissue sample. About 30 mg of soft tissue from a mouse was placed in a 5 mL microcentrifuge tube, washed in 4 mL of Cell Wash Solution (Mem-PER™ Plus Membrane Protein Extraction Kit, Thermo Scientific™), vortexed briefly and the Cell Wash Solution was discarded. The liver tissue sample was cut into small pieces and transferred to a 2 mL tissue grinder tube. 1 mL of Permeabilization Buffer (Mem-PER™ Plus Membrane Protein Extraction Kit, Thermo Scientific™) was added and the sample was homogenized to obtain an even suspension. 1 mL of Permeabilization Buffer was added to the suspension and the homogenous suspension was transferred to a new tube, and incubated for 10 minutes at 4° C. with constant mixing to release the cytosolic proteins from the permeabilized cells. The homogenous suspension of permeabilized cells was then centrifuged for 15 minutes at 16,000×g. The supernatant containing the cytosolic fraction of proteins from the liver cells were collected and transferred to a new receptacle.

To obtain the membrane fraction of proteins from the soft tissue sample, the pellet of permeabilized hepatic cells was resuspended in 1.0 mL of Solubilization Buffer (Mem-PER™ Plus Membrane Protein Extraction Kit, Thermo Scientific™) and mixed by pipetting. The suspension was then incubated for 30 minutes at 4° C. with constant mixing to release the solubilized membrane proteins into solution. The suspension was then centrifuged for 15 minutes at 16,000×g and the supernatant containing the membrane fraction of proteins from the liver cells were collected and transferred to a new receptacle.

The cytosolic fraction and membrane fraction of proteins obtained from either the adherent cell sample or soft tissue sample was subjected to bicinchoninic acid (BCA) protein assay for the colorimetric detection and quantification of total protein in each fraction according to manufacturers protocol (BCA™ Protein Assay Kit, Pierce™, the entire contents of which is hereby incorporated by reference) in order to confirm protein content in a fraction.

Protein Digestion. 800 μg of cytosolic proteins from the K562 cytosolic fraction and 400 μg of membrane proteins from the K562 cytosolic fraction obtained above were digested as follows.

Additionally, 400 μg of the cytosolic membrane proteins from the cytosolic fraction of liver tissue and 400 μg of the membrane proteins from the membrane fractions obtained from the soft murine liver tissue sample were digested according to the following protocol.

All fractions of proteins were digested by Filter Assisted Sample Preparation (FASP). Briefly, proteins in each fraction were reduced by adding 0.5M dithiothreitol (DTT) solution and incubating for 1 hour at 57° C. Microcon-30 Ultracel filters (EMD Milliporem) were equilibrated by adding 200 μl of 8M Urea solution in 100 mM Tris HC and centrifuged at 14,000×g for 15 minutes. Each protein fraction was loaded onto an appropriately labeled filter and centrifuged at 14,000×g for 15 minutes at 20° C. Next, 100 μl of 6 mM Iodoacetamide was added to each filter, mixed at 600 rpm in for 1 minute and incubated without mixing for 30 minutes in the dark. Each filter was then centrifuged at 14,000×g for 15 minutes, 100 μl of 8M Urea solution was added to each filter and each filter was centrifuged at 14,000×g for 15 minutes. This step was repeated twice. Next, 100 μl of 100 mM ammonium bicarbonate was added to each filter and each filter was centrifuged at 14,000×g for 15 minutes. This step was repeated two times. Trypsin protease was diluted in 100 mM ammonium bicarbonate to obtain an enzyme to protein ratio of 1:100. 50 μl protease solution was added to each filter and mixed at 600 rpm for 1 minute. Each filter (fraction) was then incubated overnight at room temperature to digest the membrane proteins and cytosolic proteins in their respective fractions.

Filters were then transferred to new collection tubes and centrifuged at 14,000×g for 10 minutes. Digested proteins (peptide fragments) were eluted from each filter using 50 μl of 0.5 M NaCl. Each elute was centrifuged at 14,000×g for 10 minutes. This step was repeated to increase peptide fragment yield. Peptide elutes were acidified using 0.2% trifluoroacetic acid (TFA) and desalted using C18 Sep-Pak® column chromatography (Flinn Scientific).

Labeling of Peptides. Peptides were labeled using Tandem Mass Tag™ (TMT) system (Thermo Scientific™) according to the manufacturer's protocol for quantitative analysis of glycoproteins by mass spectrometry. Briefly, 41 μL of the TMT Label Reagent (Thermo Scientific™), reconstituted in anhydrous ethanol was added to each fraction of digested peptides obtained above. The exemplary detectable marker utilized (Tandem Mass Tag) was an isobaric detectable marker, which covalently labels primary amines (—NH2 groups) of peptides. The isobaric detectable marker contains heavy isotopes, which are detectable in mass specification for sample identification and quantitation of peptides. Each mixture of label and digested peptide fraction was incubated for 1 hour at room temperature. The 8 μL of 5% hydroxylamine was added to each mixture and incubated for 15 minutes to quench the reaction.

TMT labeled cytosolic fractions of digested peptides from adherent cell samples were combined when applicable for use in certain aspects of the present methods. TMT labeled membrane fractions of digested peptides from adherent cell samples were combined when applicable for use in certain aspects of the present methods.

TMT labeled cytosolic fractions of digested peptides from soft tissue samples obtained from mouse liver were combined when applicable for use in certain aspects of the present methods. TMT labeled membrane fractions of digested peptides from soft tissue samples obtained from mouse liver were combined when applicable for use in certain aspects of the present methods.

After incubation, each labeled digested peptide fraction was desalted using C18 Sep-Pak® column chromatography (Flinn Scientific) and excess label was removed.

Fractionation of cytosolic and membrane digested peptides by ion-pairing hydrophilic interaction liquid chromatography (HILIC). Here, digested peptides from cytosolic peptide fragment samples or membrane peptide fragment samples were fractionated individually on a TSKgel® Amide-80 HR HPLC column (Sigma Aldrich®) using an Acquity ultra performance liquid chromatography (UPLC) system with fraction collector (ACQUITY UPLC® System, Waters Inc.) according to manufacturer's protocol. Fractions of cytosolic or membrane peptides were collected every one minute throughout gradient separation. Fractions 19-36 for each cytosolic and membrane sample of digested peptides were enriched in glycosylated peptide fragments, and thus separated for further analysis.

Mass spectrometry and glycoproteomic spectra analysis. Each fraction of peptide fragments enriched in glycosylated peptides was loaded onto a 25 cm Acclaim™ PepMap™ C18 liquid chromatography column (Thermo Scientific™) using UltiMate™ 3000 RSLCnano (Thermo Scientific™) low flow liquid chromatography system and eluted into Q Exactive™ HF-X mass spectrometer (ThermoScientific™).

Raw mass spectral data for each fraction of glycosylated peptide fragments was compared against Byonic™ mass spectrometry search engine and database using Proteome Discoverer™ 2.2. software (Thermo Scientific™) to identify and quantify glycoproteins. For analysis, peptide mass tolerance was kept to 10 ppm for MS1 and 20 ppm for MS2.

For human cell samples such as the above adherent K562 samples, mass spectral data was searched against the Uniprot Human proteome database and the Byonic™ human glycan database was used to identify glycopeptides, PSM, glycoproteins, glycan composition and glycosylation sites in each fraction.

For murine cell samples such as the above mouse liver tissue samples, mass spectral data was searched against the Uniprot mouse proteome database and the Byonic™ mammalian glycan database was used to identify glycopeptides, PSM, glycoproteins, glycan composition and glycosylation sites in each fraction. In each instance, peptides identified with a Byonic™ peptide score <300 and Byonic™ Log Probability score <2 were excluded.

### Example 2. Whole-Cell Glycoprotein Profiling of Adherent Human Cells

Human K562 bone marrow cells (ATCC® CCL-243™) were grown to confluence and a sample containing 2.5×10⁶ cells were processed as stated in Example 1 above to obtain a cytosolic fraction of proteins from the cells and a membrane fraction of proteins from the cells. Each of the cytosolic fraction of proteins and membrane fraction of proteins were then digested and isobarically labeled as indicated above to generate a cytosolic fraction of peptide fragments from the cell sample and a membrane fraction of peptide fragments from the cell sample.

Each fraction of cytosolic and/or membrane peptide fragments were enriched by separating non-glycosylated peptides from the fractions and fractionated by ion-pair HILIC as indicated above in Example 1. Fractions 19-36 were isolated glycans were removed from the enriched glycoproteins using a glycosidase, e.g., an amidase such as PNGaseF.

LC-MS was performed on each fraction to obtain mass spectral data for the cytosolic fraction and membrane fraction. The mass spectral data was further analyzed using the Byonic human glycan database and search engine, then compared to the UNIPROT human proteome database to obtain the glycoprotein profile of the cytosolic fraction of human cells from the sample, the glycoprotein profile of the membrane fraction of human cells from the sample and whole-cell glycoprotein profile.

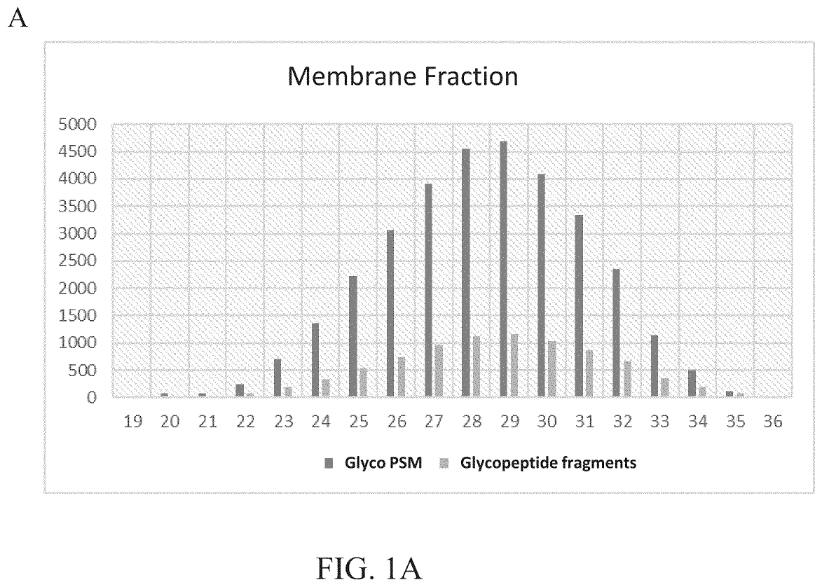

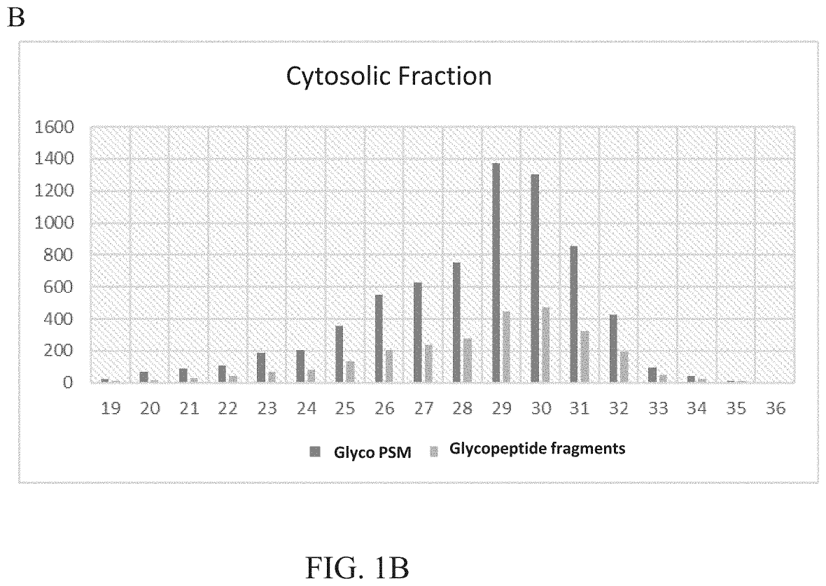

LC-MS data was evaluated against the human protein database to generate a peptide-spectrum match (PSM), which was used to identify the peptide present in the sample. As shown in FIG. 1A, as well as Table 1 below, the total number glycopeptides fragments were identified from the membrane fraction of K562 cells and the PSM was determined for each glycopeptide identified by the mass spectra for each fraction (19-36) analyzed.

TABLE 1
Glycopeptide fragments identified by LC-MS for each fraction of the
membrane protein fraction analyzed and the corresponding PSM.
PSM (total number of identified peptide spectra matched to the
glycopeptides fragment) value is higher than total number of
glycopeptides fragments identified in each fraction,
indicating that glycopeptides were identified repeatedly.

| Fraction | | Glycopeptide |
|---|---|---|
| Number | PSM | fragment |
| 19 | 20 | 7 |
| 20 | 78 | 9 |
| 21 | 76 | 26 |
| 22 | 248 | 73 |
| 23 | 695 | 187 |
| 24 | 1369 | 336 |
| 25 | 2217 | 530 |
| 26 | 3065 | 741 |
| 77 | 3907 | 964 |
| 78 | 4545 | 1120 |
| 29 | 4703 | 1166 |
| 30 | 4085 | 1025 |
| 31 | 3341 | 861 |
| 32 | 2352 | 670 |
| 33 | 1137 | 362 |
| 34 | 505 | 192 |
| 35 | 108 | 76 |
| 36 | 6 | 4 |

Table 2 below, shows that the present methods can be used to identify the glycoproteins present in the membrane fraction of a sample. Furthermore, the abundance of each glycoprotein is identified based on PSM score.

TABLE 2
List of fifty (50) most abundant glycoproteins present in the
membrane fraction of K562 cells according to PSM.

| Protein Name | # PSMs |
|---|---|
| Hypoxia up-regulated protein 1 | 2062 |
| Isoform LAMP-2C of Lysosome- | 1759 |
| associated membrane glycoprotein 2 | |
| Cation-independent mannose-6-phosphate receptor | 1682 |
| Lysosome-associated membrane glycoprotein 1 | 1627 |
| Basigin | 939 |
| Transferrin receptor protein 1 | 852 |
| Isoform 3 of Calumenin | 727 |
| Prolyl 4-hydroxylase subunit alpha-1 | 682 |
| Transmembrane 9 superfamily member 3 | 658 |
| Isoform 3 of Integrin beta-1 | 648 |
| Translocon-associated protein subunit alpha | 606 |
| Endoplasmin | 582 |
| Isoform Sap-mu-9 of Prosaposin | 563 |
| Receptor-type tyrosine-protein phosphatase C | 548 |
| Synaptophysin-like protein 1 | 472 |
| 4F2 cell-surface antigen heavy chain | 451 |
| Cleft lip and palate transmembrane protein | 435 |
| 1-like protein | |
| Procollagen galactosyltransferase 1 | 406 |
| Sortilin | 387 |
| Nicastrin | 382 |
| Prenylcysteine oxidase 1 | 360 |
| Dolichyl-diphosphooligosaccharide— | 348 |
| protein glycosyltransferase subunit STT3B | |
| Integrin alpha-5 | 342 |
| Palmitoyl-protein thioesterase 1 | 339 |
| Nuclear pore membrane glycoprotein 210 | 321 |
| Protein sel-1 homolog 1 | 319 |
| Uncharacterized protein | 312 |
| Carboxypeptidase D | 308 |
| Glycophorin-A | 286 |
| Transforming growth factor beta-1 proprotein | 286 |
| Transport and Golgi organization protein 1 homolog | 282 |
| Leukocyte surface antigen CD47 (Fragment) | 278 |
| Sodium/potassium-transporting ATPase subunit beta-3 | 269 |
| Isoform A of Leptin receptor | 267 |
| Multifunctional procollagen lysine hydroxylase | 254 |
| and glycosyltransferase LH3 | |
| Isoform 3 of Prolyl 3-hydroxylase 1 | 251 |
| Adipocyte plasma membrane-associated protein | 249 |
| UDP-glucose:glycoprotein glucosyltransferase 1 | 222 |
| Disintegrin and metalloproteinase | 221 |
| domain-containing protein 17 | |
| Cation-dependent mannose-6-phosphate receptor | 219 |
| Ceramide synthase 2 | 216 |
| Dipeptidyl peptidase 1 | 203 |
| STIM1L | 203 |
| Nodal modulator 3 | 201 |
| Transmembrane protein 106B | 201 |
| Disintegrin and metalloproteinase | 199 |
| domain-containing protein 10 | |
| Plexin-B2 | 197 |
| GPI transamidase component PIG-T | 196 |
| Vitronectin | 185 |
| Isoform 3 of Golgi apparatus protein 1 | 175 |

In addition FIG. 1B and Table 3 below, show the total number glycopeptide fragments were identified from the cytosolic fraction of K562 cells and the PSM for each glycopeptide identified by the mass spectra for each cytosolic peptide fraction (19-36) analyzed.

TABLE 3
Glycopeptide fragments identified by LC-MS for each fraction of the
cytosolic protein fraction analyzed and the corresponding PSM.

| Fraction | Glyco | Glycoprotein |
|---|---|---|
| Number | PSM | fragments |
| 19 | 27 | 11 |
| 20 | 69 | 16 |
| 21 | 92 | 30 |
| 22 | 107 | 43 |
| 23 | 184 | 68 |
| 24 | 207 | 80 |
| 25 | 352 | 137 |
| 26 | 553 | 205 |
| 27 | 628 | 241 |
| 28 | 752 | 279 |
| 29 | 1372 | 446 |
| 30 | 1301 | 473 |
| 31 | 856 | 320 |
| 32 | 428 | 193 |
| 33 | 96 | 52 |
| 34 | 42 | 25 |
| 35 | 10 | 11 |
| 36 | 2 | 5 |

Table 4 shows that the present methods can be used to identify the glycoproteins present in the cytosolic fraction of a cell sample. Again, the abundance of each glycoprotein is identified based on PSM score.

TABLE 4
List of fifty (50) most abundant glycoproteins present in the cytosolic
fraction of K562 cells according to PSM.

| Protein Name | #PSMs |
|---|---|
| Hypoxia up-regulated protein 1 | 843 |
| Dipeptidyl peptidase 1 | 405 |
| Isoform Sap-mu-9 of Prosaposin | 361 |
| Palmitoyl-protein thioesterase 1 | 328 |
| Isoform LAMP-2C of Lysosome-associated | 322 |
| membrane glycoprotein 2 | |
| Lysosome-associated membrane glycoprotein 1 | 312 |
| Isoform 3 of Calumenin | 290 |
| Cathepsin D | 197 |
| Prolyl 4-hydroxylase subunit alpha-1 | 182 |
| Serpin H1 | 164 |
| Protein CREG1 | 159 |
| Beta-galactosidase | 157 |
| Phospholipase D3 | 152 |
| STON1-GTF2A1L readthrough | 147 |
| Endoplasmin | 146 |
| Gamma-glutamyl hydrolase | 139 |
| N-acetylglucosamine-6-sulfatase | 138 |
| Transferrin receptor protein 1 | 128 |
| Cation-independent mannose-6-phosphate receptor | 122 |
| Metalloproteinase inhibitor 1 | 100 |
| Prolyl 3-hydroxylase 1 | 96 |
| UDP-glucose:glycoprotein glucosyltransferase 1 | 85 |
| Cathepsin L1 | 81 |
| Carboxypeptidase | 75 |
| Transmembrane 9 superfamily member 3 | 75 |
| Isoform 4 of Calumenin | 69 |
| Acid ceramidase (Fragment) | 66 |
| Transforming growth factor beta-1 proprotein | 63 |
| Multifunctional procollagen lysine hydroxylase | 56 |
| and glycosyltransferase LH3 | |
| Polycystic kidney disease 2-like 2 protein | 54 |
| Translocon-associated protein subunit alpha | 54 |
| Sortilin | 49 |
| Cartilage-associated protein | 47 |
| Cleft lip and palate transmembrane protein 1-like protein | 46 |
| Basigin | 45 |
| Synaptophysin-like protein 1 | 45 |
| Beta-hexosaminidase subunit beta | 44 |
| Ribonuclease T2 | 42 |
| Beta-hexosaminidase | 39 |
| Lysosomal acid phosphatase | 39 |
| Tripeptidyl-peptidase 1 | 38 |
| Disintegrin and metalloproteinase | 37 |
| domain-containing protein 10 | |
| Microfibril-associated glycoprotein 4 | 37 |
| Glycophorin | 36 |
| Torsin-4A | 35 |
| Transport and Golgi organization protein 1 homolog | 35 |
| Sodium/potassium-transporting ATPase subunit beta-3 | 34 |
| Sialidase-1 | 31 |
| Alpha-galactosidase | 30 |
| Isoform 6 of Cysteine-rich with EGF-like domain protein 2 | 30 |

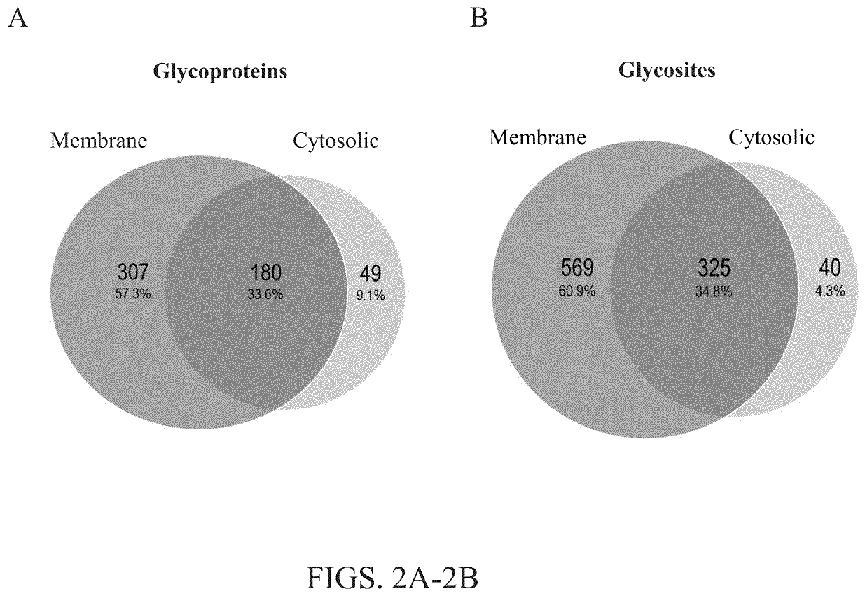

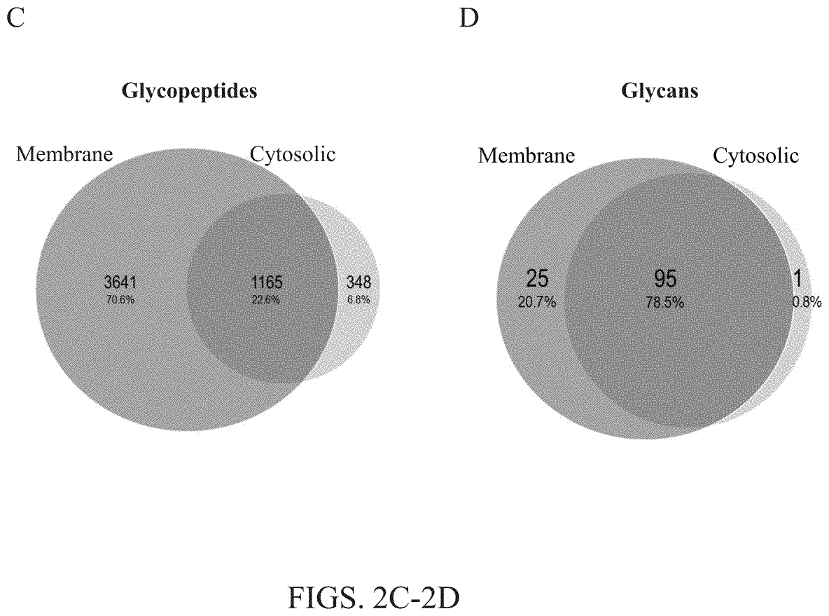

The mass spectral data for the membrane and cytosolic fractions of the human K562 cell sample were then compared to quantitatively identify the total number of glycosylation sites (glycosites), glycopeptides fragments (glycopeptides), glycan composition (glycans) and glycoproteins in each of the cytosolic fraction and membrane fraction. See FIGS. 2A-2D and Table 5 below.

TABLE 5
Quantitative whole-cell glycoproteomic
analysis of human cells.

| | Glyco- | Glyco- | Glyco- | Glycan |
|---|---|---|---|---|
| Samples | protein | site | peptides | Composition |
| Membrane | 487 | 894 | 4806 | 120 |
| Fraction | | | | |
| Cytosolic | 229 | 365 | 1513 | 96 |
| Fraction | | | | |
| Whole-cell | 536 | 934 | 5154 | 121 |
| Unique | | | | |

The data shows that the present methods successfully identified 365 glycosylation sites, 1513 glycopeptide fragments, 229 glycoproteins, and 96 glycans in the cytosolic fraction of K562 cells and 894 glycosylation sites, 4806 glycopeptide fragments, 487 glycoproteins and 120 glycans were identified from the membrane fraction of K562 cell line.

Furthermore, of the 894 glycosylation sites identified in the membrane fraction and the 365 identified in the cytosolic fraction of K562 cells, 83% of the glycosylation sites in each fraction (i.e., 740 and 303, respectively) were verified by deglycosylation of individual HILIC fractions and LCMS analysis.

Additionally, a further analysis of the spectral data reveal a total of 934 unique glycosylation sites, 5154 unique glycopeptide fragments, 536 unique glycoproteins, and 121 of the possible 132 human glycans were identified in the whole-cell (combining cytosolic fraction identification and membrane fraction identification) as shown in FIGS. 2A-2D.

### Example 3. Whole-Cell Glycoprotein Profiling of Soft Tissue from Mice

Murine liver tissue was obtained and a 30 mg soft tissue sample was homogenized, and processed as stated in Example 1 above to obtain a cytosolic fraction of proteins from the liver cells and a membrane fraction of proteins from the liver cells. Each of the cytosolic fraction of proteins and membrane fraction of proteins were then digested and isobarically labeled as indicated above to generate a cytosolic fraction of peptide fragments from the cell sample and a membrane fraction of peptide fragments from the cell sample.

Each fraction of cytosolic and/or membrane peptide fragments were enriched by removing non-glycosylated peptides from the fractions and fractionated by ion-pairing HILIC as indicated above in Example 1 and 2. Fractions 19-36 were isolated glycans were removed from the enriched glycoproteins using a glycosidase, e.g., the amidase, PNGaseF.

LC-MS was performed on each fraction to obtain mass spectral data for the cytosolic fraction and membrane fraction. The mass spectral data was further analyzed using the Byonic™ mammalian glycan database and search engine, then compared to the Uniprot mouse proteome database to obtain the glycoprotein profile of the cytosolic fraction of murine liver cells from the sample, the glycoprotein profile of the membrane fraction of human cells from the sample and whole-cell glycoprotein profile.

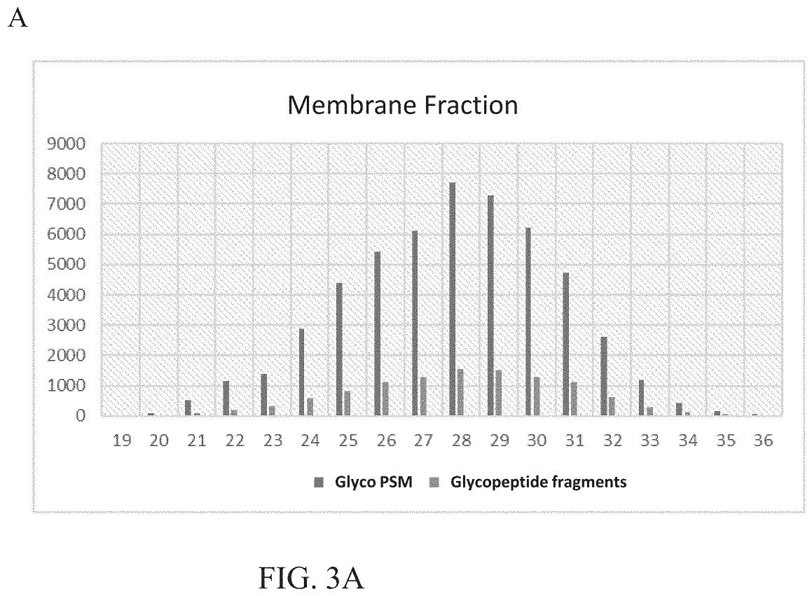

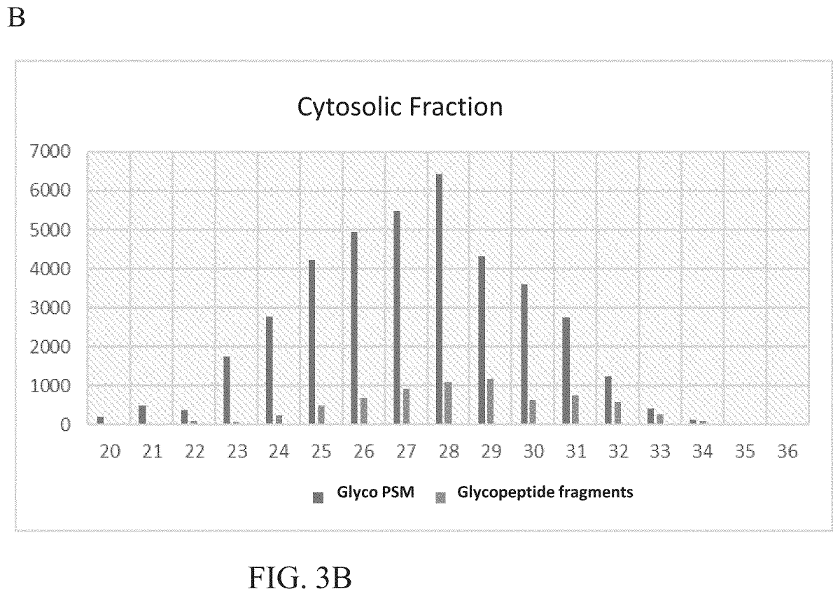

LC-MS data was evaluated against the murine protein database to generate a peptide-spectrum match (PSM), which was used to identify the peptide present in the sample. As shown in FIG. 3A, as well as Table 6 below, the total number glycopeptides fragments were identified from the membrane fraction of mouse liver cells and the PSM was determined for each glycopeptide identified by the mass spectra for each fraction (19-36) analyzed.

TABLE 6
Glycopeptide fragments identified by LC-MS for each fraction of the
membrane protein fraction analyzed and the corresponding PSM.
PSM (total number of identified peptide spectra matched to the
glycopeptides fragment) value is higher than total number of
glycopeptides fragments identified in each fraction, indicating that
glycopeptides were identified repeatedly.

| Fraction | Glyco | Glycopeptide |
|---|---|---|
| Number | PSM | fragments |
| 19 | 40 | 16 |
| 20 | 100 | 23 |
| 21 | 523 | 81 |
| 22 | 1140 | 183 |
| 23 | 1392 | 319 |
| 24 | 2891 | 592 |
| 25 | 4387 | 816 |
| 26 | 5409 | 1125 |
| 27 | 6136 | 1280 |
| 28 | 7716 | 1563 |
| 29 | 7282 | 1515 |
| 30 | 6223 | 1291 |
| 31 | 4724 | 1122 |
| 32 | 2602 | 621 |
| 33 | 1182 | 290 |
| 34 | 413 | 114 |
| 35 | 162 | 49 |
| 36 | 47 | 27 |

Table 7 below, shows that the present methods can be used to identify the glycoproteins present in the membrane fraction of a soft tissue sample. Furthermore, the abundance of each glycoprotein is identified based on PSM score.

TABLE 7
List of fifty (50) most abundant glycoproteins present in the
membrane fraction of mouse liver tissue cells according to PSM.

| Protein Name | #PSMs |
|---|---|
| Dipeptidyl peptidase 4 | 1896 |
| Aminopeptidase N | 1771 |
| Prenylcysteine oxidase | 1704 |
| Low density lipoprotein receptor-related protein 1 | 1648 |
| CEA-related cell adhesion molecule 1 | 1519 |
| Isoform LAMP-2B of Lysosome-associated | 1311 |
| membrane glycoprotein 2 | |
| UDP-glucuronosyltransferase 1-1 | 1219 |
| Tripeptidyl-peptidase 1 | 1181 |
| Carboxylesterase 3A | 1136 |
| Lysosome-associated membrane glycoprotein 1 | 1136 |
| Corticosteroid 11-beta-dehydrogenase isozyme 1 | 1100 |
| Pyrethroid hydrolase Ces2a | 1053 |
| N-fatty-acyl-amino acid synthase/ | 1044 |
| hydrolase PM20D1 | |
| Murinoglobulin-1 | 1032 |
| Hypoxia up-regulated protein 1 | 859 |
| Carboxylesterase 1D | 852 |
| Lysosomal acid lipase/cholesteryl ester hydrolase | 821 |
| Integrin alpha-1 | 797 |
| Scavenger receptor class B member 1 | 770 |
| Platelet glycoprotein 4 | 697 |
| H-2 class I, histocompatibility | 645 |
| antigen, K-B alpha chain | |
| Endoplasmin | 613 |
| Low affinity immunoglobulin | 585 |
| gamma Fc region receptor II | |
| Carboxylesterase 1F | 565 |
| Serine protease inhibitor A3K | 565 |
| Immunoglobulin heavy constant mu (Fragment) | 551 |
| Lysosome membrane protein 2 | 551 |
| Carboxypeptidase | 527 |
| Isoform 2 of Integrin beta-1 | 521 |
| Arylacetamide deacetylase | 508 |
| UDF-glucuronosyltransferase 2A3 | 502 |
| Haptoglobin | 484 |
| UDP-glucuronosyltransferase 3A2 | 484 |
| Carboxylesterase 3B | 440 |
| Serum paraoxonase/arylesterase 1 | 436 |
| Cation-dependent mannose-6-phosphate receptor | 428 |
| Cell adhesion molecule 1 | 427 |
| Plexin-B2 | 404 |
| Basigin | 400 |
| ADP-ribosyl cyclase/cyclic ADP- | 373 |
| ribose hydrolase 1 | |
| Translocon-associated protein | 372 |
| subunit beta (Fragment) | |
| Kininogen-1 | 361 |
| Acid ceramidase | 339 |
| Major urinary protein 3 | 325 |
| Carboxylic ester hydrolase | 314 |
| GDH/6PGL endoplasmic bifunctional protein | 314 |
| Pregnancy zone protein | 309 |
| Prosaposin | 308 |
| Protein sel-1 homolog 1 | 307 |
| Thioredoxin domain-containing protein 15 | 306 |

In addition FIG. 3B and Table 8 below, identify the total number glycopeptides fragments detected in the cytosolic fraction of mouse liver tissue cells and the PSM for each glycopeptide identified by the mass spectra for each cytosolic peptide fraction (19-36) analyzed.

TABLE 8
Glycopeptide fragments identified by LC-MS for each fraction of the
cytosolic protein fraction analyzed and the corresponding PSM.

| Fraction | Glyco | Glycopeptide |
|---|---|---|
| Number | PSM | fragments |
| 19 | 37 | 9 |
| 20 | 193 | 39 |
| 21 | 493 | 95 |
| 22 | 387 | 75 |
| 23 | 1745 | 231 |
| 24 | 2761 | 493 |
| 25 | 4216 | 686 |
| 26 | 4935 | 924 |
| 27 | 5470 | 1097 |
| 28 | 6429 | 1172 |
| 29 | 4301 | 622 |
| 30 | 3601 | 755 |
| 31 | 2739 | 575 |
| 32 | 1233 | 253 |
| 33 | 405 | 100 |
| 34 | 134 | 50 |
| 35 | 24 | 9 |
| 36 | 23 | 5 |

Table 9 shows that the present methods can be used to identify the glycoproteins present in the cytosolic fraction of a tissue sample containing cells. Again, the abundance of each glycoprotein is identified based on PSM score.

TABLE 9
List of fifty (50) most abundant glycoproteins present in the cytosolic
fraction of liver cells obtained from soft tissue according to PSM.

| Protein Name | # PSMs |
|---|---|
| Carboxylesterase 3A | 1994 |
| Murinoglobulin-1 | 1664 |
| Pregnancy zone protein | 1264 |
| Tripeptidyl-peptidase 1 | 1237 |
| Hypoxia up-regulated protein 1 | 1065 |
| Pyrethroid hydrolase Ces2a | 1009 |
| Immunoglobulin heavy constant mu (Fragment) | 972 |
| Carboxypeptidase | 914 |
| MCG1051009 | 903 |
| Endoplasmin | 901 |
| Haptoglobin | 835 |
| Prolow-density lipoprotein receptor-related protein 1 | 818 |
| Alpha-1-antitrypsin 1-4 | 808 |
| Carboxylesterase 1D | 739 |
| Cathepsin D | 727 |
| Major urinary protein 3 | 702 |
| Lysosomal acid lipase/cholesteryl ester hydrolase | 634 |
| Carboxylesterase 3B | 632 |
| Isoform LAMP-2B of Lysosome- | 610 |
| associated membrane glycoprotein 2 | |
| Carboxylesterase 1F | 547 |
| Fibrinogen beta chain | 518 |
| Biotinidase | 496 |
| Carboxypeptidase Q | 496 |
| Predicted gene 20425 | 486 |
| Protein disulfide-isomerase A2 | 472 |
| Carboxylic ester hydrolase | 445 |
| GDH/6PGL endoplasmic bifunctional protein | 409 |
| Lysosome-associated membrane glycoprotein 1 | 408 |
| Group XV phospholipase A2 | 396 |
| Lysosomal alpha-glucosidase | 388 |
| Kininogen-1 | 378 |
| Pyrethroid hydrolase Ces2e | 372 |
| Cathepsin Z | 362 |
| Prenylcysteine oxidase | 362 |
| Zinc-alpha-2-glycoprotein | 361 |
| Prosaposin | 353 |
| Lysosomal alpha-mannosidase | 346 |
| UDP-glucuronosyltransferase 1-1 | 340 |
| Arylacetamide deacetylase | 330 |
| Carboxylesterase 3B (Fragment) | 330 |
| Carboxylesterase 1E | 285 |
| N-acetylglucosamine-6-sulfatase | 280 |
| Liver carboxylesterase 1 | 278 |
| Ectonucleoside triphosphate diphosphohydrolase 5 | 277 |
| Putative phospholipase B-like 2 | 236 |
| Cation-dependent mannose-6-phosphate receptor | 234 |
| Cathepsin L1 | 230 |
| Heat shock 70 kDa protein 1-like | 216 |
| Endoplasmic reticulum aninopeptidase 1 | 212 |
| Alpha-1-acid glycoprotein 1 | 207 |

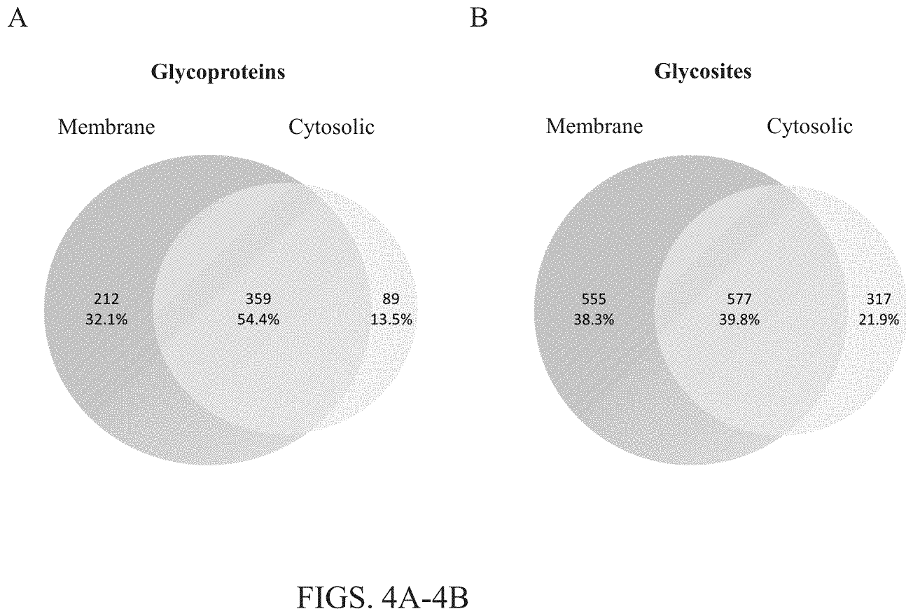

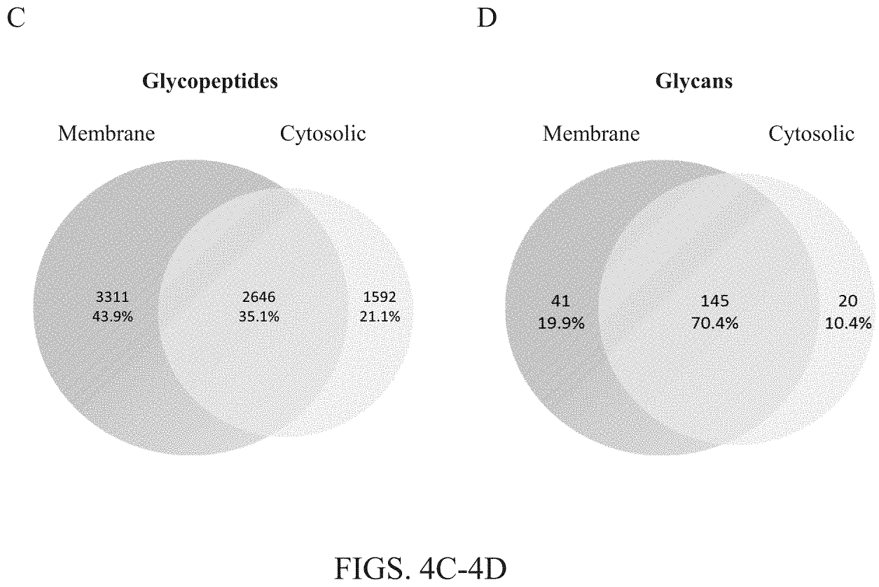

The mass spectral data for the membrane and cytosolic fractions of the murine hepatic cells from a soft tissue sample were then compared in order to quantitatively identify the total number of glycosylation sites (glycosites), glycopeptide fragments (glycopeptides), glycan composition (glycans) and glycoproteins in each of the cytosolic fraction and membrane fraction. See FIGS. 4A-4D and Table 10 below.

TABLE 10
Quantitative whole-cell glycoproteomic
analysis of murine cells.

| | Glyco- | Glyco- | Glycan | Glyco- |
|---|---|---|---|---|
| Samples | protein | site | Composition | peptides |
| Membrane | 571 | 1132 | 186 | 5957 |
| Fraction | | | | |
| Cytosolic | 448 | 894 | 165 | 4238 |
| Fraction | | | | |
| Whole-cell | 660 | 1449 | 206 | 7549 |
| Unique | | | | |

The data shows that the present methods successfully identified 894 glycosylation sites, 4238 glycopeptide fragments, 448 glycoproteins, and 165 glycans in the cytosolic fraction of murine liver cells and 1132 glycosylation sites, 5957 glycopeptide fragments, 571 glycoproteins and 186 glycans were identified from the membrane fraction of the murine liver cells.

Additionally, a further analysis of the spectral data reveal a total of 1449 unique glycosylation sites, 7549 unique glycopeptide fragments, 660 unique glycoproteins, and 206 of the possible 304 mammalian glycans were identified in the whole-cell (combining cytosolic fraction identification and membrane fraction identification) as shown in FIGS. 4A-4D.

Taken together, the data herein show that the present methods can be used to generate a complete analysis of compartmentalized glycosylation of proteins independent of species or type of sample from which the cells are obtained. Therefore, the present methods provide a whole-cell analysis of glycosylation in any biological system and enables quantitation of glycosylation.

### Example 4: Reproducibility of Processing Human Adherent Cells to Obtain Membrane and Cytosolic Protein Fractions

Human K562 bone marrow cells (ATCC® CCL-243™) were grown to confluence and a sample containing 2.5×10⁶ cells were processed as stated in Example 1 above to obtain 2 replicate cytosolic fractions of proteins from the cells and 2 replicate membrane fractions of proteins from the cells. Each replicate fraction from (cytosolic and membrane) human K562 cell samples were digested separately by Filter Assisted Sample Preparation (FASP) as set forth in Example 1, above. The resulting fractions of cytosolic peptide fragments and membrane peptide fragments were then labeled with an isobaric detectable marker using the Tandem Mass Tag™ (TMT) system (Thermo Scientific™), as set forth in Example 1. The labeled cytosolic peptide fragments from the cytosolic replicates were collected and combined to create a mixture of labeled cytosolic peptide fragments from both replicate fractions. The labeled membrane peptide fragments from the membrane replicates were collected and combined to create a mixture of labeled membrane peptide fragments from both replicate membrane fractions.

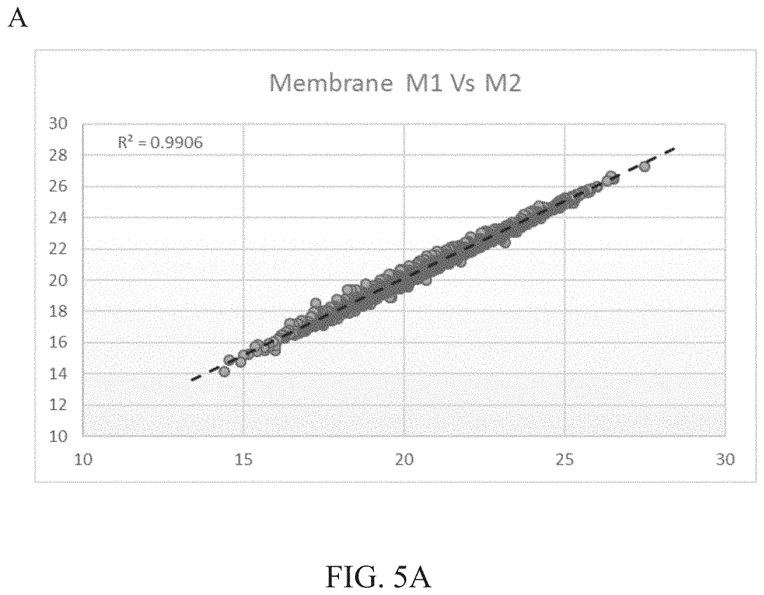

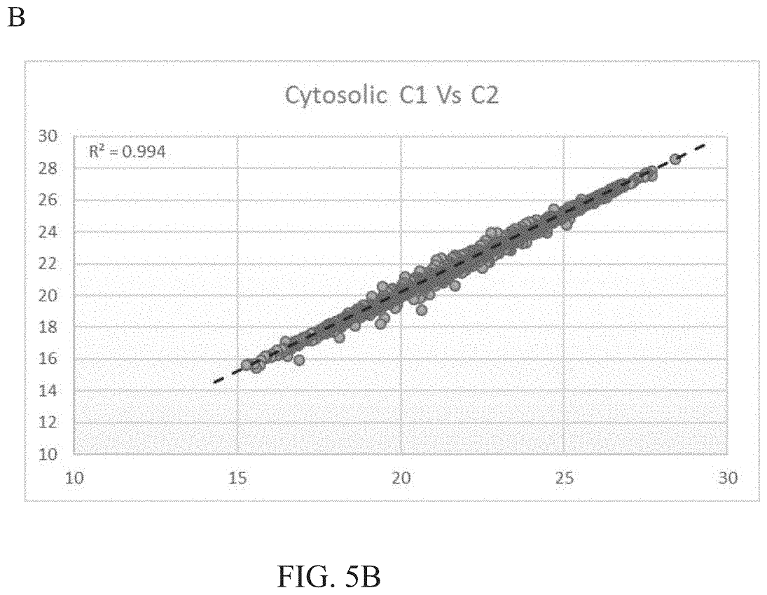

Liquid chromatography mass spectrometry was then used to measure intensity of detectable marker generated signals (i.e., TMT reporter ions) of all membrane peptide fragments in the replicate membrane fractions present in the replicate preparations of membrane fractions from human K562 cells as were all cytosolic peptide fragments in the replicate cytosolic fractions present in the replicate preparations of cytosolic fractions from human K562 cells. See FIGS. 5A and 5B. FIGS. 5A and 5B show scatter plots of reporter ion intensities from all proteins in membrane fraction replicates (M1 and M2) and cytosolic fraction replicates (C1 and C2) obtained from human K562 adherent cells detected in the HCD MS/MS spectra.

The linear relationship between both cytosolic and membrane replicate preparations show a correlation coefficients (R2) of greater than 0.99 for each of the membrane and cytosolic preparations. These data show that the processing methods for the obtaining of cytosolic fractions and membrane fractions of proteins from adherent cells are highly consistent and reproducible.

### Example 5: Reproducibility of Processing Murine Liver Tissue Samples to Obtain Membrane and Cytosolic Protein Fractions

Murine soft liver tissue samples were homogenized and processed as set forth above in Example 1 to obtain 2 replicate cytosolic fractions of proteins from the murine liver cells and 2 replicate membrane fractions of proteins from the murine liver cells. As stated above in Example 4, each replicate fraction from (cytosolic and membrane) murine tissue samples were digested separately by Filter Assisted Sample Preparation (FASP). The resulting fractions of cytosolic peptide fragments and membrane peptide fragments were then labeled using the Tandem Mass Tag™ (TMT) system (Thermo Scientific™). The labeled cytosolic peptide fragments from the cytosolic replicates were collected and combined to create a mixture of labeled cytosolic peptide fragments from both replicate fractions. The labeled membrane peptide fragments from the membrane replicates were collected and combined to create a mixture of labeled membrane peptide fragments from both replicate membrane fractions.

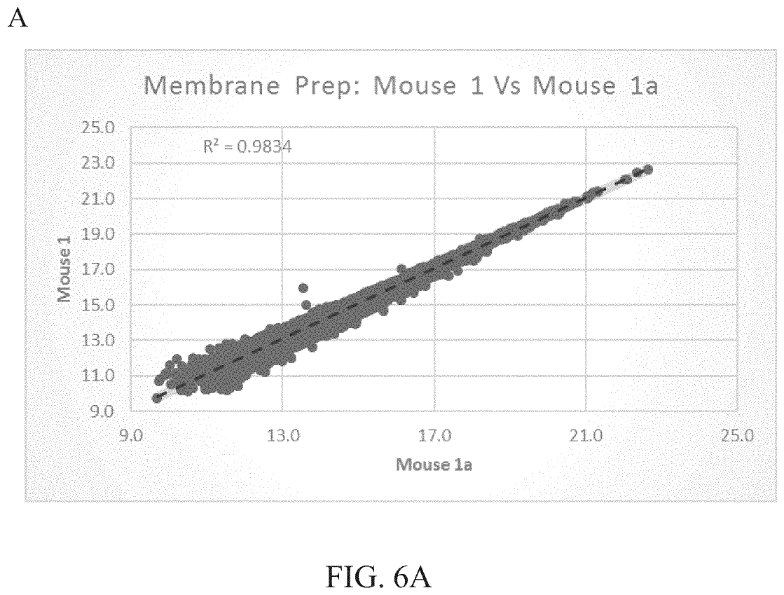

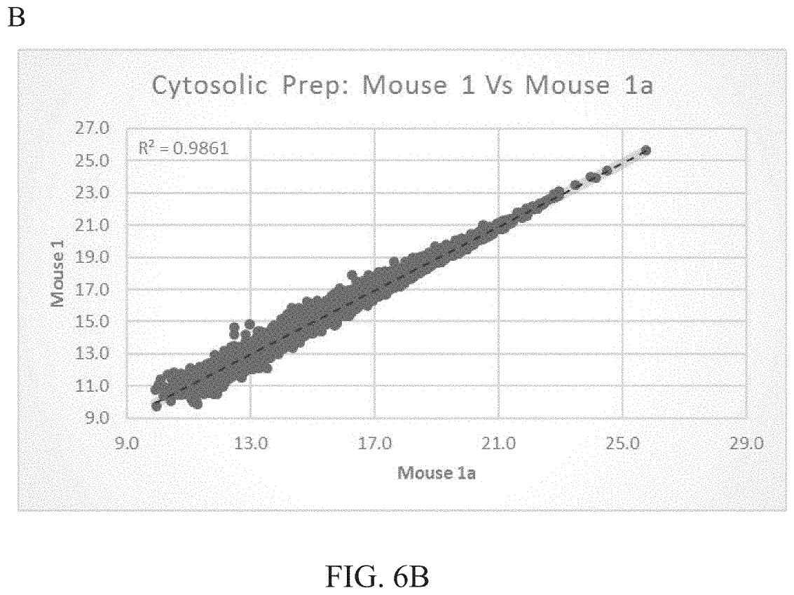

Liquid chromatography mass spectrometry was then used to measure intensity of detectable marker generated signals of all membrane peptide fragments in the replicate membrane fractions present in the replicate preparations of membrane fractions from murine tissue cells as were all cytosolic peptide fragments in the replicate cytosolic fractions present in the replicate preparations of cytosolic fractions of the murine tissue cells. See FIGS. 6A and 6B.

FIGS. 6A and 6B show scatter plots of reporter ion intensities detected in the HCD MS/MS spectra of all proteins in membrane fraction replicates (M1 and M2) and cytosolic fraction replicates (C1 and C2) obtained from liver cells isolated from murine liver tissue samples. The Linear relationship between both cytosolic and membrane replicate preparations show a correlation coefficients (R2) of greater than 0.98 for each of the membrane and cytosolic preparations. These data show that the processing methods for the obtaining of cytosolic fractions and membrane fractions of proteins from soft tissue samples are also highly consistent and reproducible.

1. A method for profiling of glycoproteins comprising:
(a) processing a sample comprising cells to isolate a cytosolic fraction of the cells and a membrane fraction of the cells, and
(b) performing a mass spectrometry analysis of the proteins in the membrane fraction to obtain a profile of glycoproteins in the membrane fraction, and performing a mass spectrometry analysis of the proteins in the cytosolic fraction to obtain a profile of glycoproteins in the cytosolic fraction.

2. The method of claim 1, wherein said processing comprises:
(i) mixing the cells from the sample with a permeabilization solution comprising a first detergent to permeabilize the plasma membrane of the cells in the sample;
(ii) subjecting the mixture from step (i) to centrifugation to obtain a first pellet comprising permeabilized cells, and a supernatant comprising the cytosolic fraction;
(iii) collecting the supernatant from step (ii), and suspending the first pellet from step (ii) in a solubilization solution comprising a second detergent to form a suspension, wherein the second detergent that solubilizes membrane proteins from the cells;
(iv) subjecting the suspension from step (iii) to centrifugation to obtain a second pellet and a supernatant comprising the membrane fraction; and
(v) collecting the supernatant from step (iv).

3. The method of claim 2, wherein the solubilization solution comprises an ionic detergent.

4. The method of claim 3, wherein the ionic detergent is selected from the group consisting of sodium dodecyl sulfate (SDS), sodium deoxycholate, N-lauryl sarcosine, 3-[(3-cholamidopropyl)dimethylammonio]-1-propanesulfonate (CHAPS) and a combination thereof.

5. The method of claim 3, wherein the solubilization solution comprises the ionic detergent at a concentration of 0.1% to 1.0% weight by volume.

6. The method of claim 2, wherein the permeabilization solution comprises a nonionic detergent.

7. The method of claim 6, wherein the nonionic detergent is selected from the group consisting of Triton-X 100, octylphenoxypolyethoxyethanol, polysorbate 20 (Tween-20), Saponin and a combination thereof.

8. The method of claim 6, wherein the permeabilization solution comprises the nonionic detergent at a concentration of 0.1%-0.2% weight by volume.

9. The method of claim 1, wherein the profile of glycoproteins in the membrane fraction is obtained by a process comprising:
(1) digesting proteins in the membrane fraction to obtain peptide fragments;
(2) separating non-glycosylated peptide fragments from the peptide fragments of step (1) to obtain peptide fragments enriched in glycosylated peptides; and
(3) performing a mass spectrometry analysis of the peptide fragments enriched in glycosylated peptides obtained in step (2), to obtain the profile of glycoproteins in the membrane fraction, wherein the profile comprises a listing of glycoproteins, optionally with one or more of glycosylation sites, glycopeptides, glycan composition, and abundance of the glycoproteins.

10. The method of claim 1, wherein the profile of glycoproteins in the cytosolic fraction is obtained by a process comprising:
(1) digesting proteins in the cytosolic fraction to obtain peptide fragments;
(2) separating non-glycosylated peptide fragments from the peptide fragments of step (1) to obtain peptide fragments enriched in glycosylated peptides; and
(3) performing a mass spectrometry analysis of the peptide fragments enriched in glycosylated peptides obtained in step (2), to obtain the profile of glycoproteins in the cytosolic fraction, wherein the profile comprises a listing of glycoproteins, optionally with one or more of glycosylation sites, glycosylated peptides, glycan composition, and abundance of the glycoproteins.

11.-12. (canceled)

13. The method of claim 9, wherein said separating the non-glycosylated peptide fragments of the membrane fraction in step (2) comprises performing ion-pairing hydrophilic interaction liquid chromatography, lectin affinity chromatography, or hydrazide capture.

14. The method of claim 10, wherein said separating the non-glycosylated peptide fragments of the cytosolic fraction in step (2) comprises performing ion-pairing hydrophilic interaction liquid chromatography, lectin affinity chromatography, or hydrazide capture.

15. The method of claim 1, wherein the cells are mammalian cells.

16. (canceled)

17. The method of claim 2, wherein the sample of step (a) is a tissue sample, and the processing step further comprises, prior to step (a)(i) homogenizing the tissue sample.

18. (canceled)

19. The method of claim 9, wherein the peptide fragments enriched in glycosylated peptides from the membrane fraction are treated with a glycosidase to release glycans.

20. The method of claim 10, wherein the peptide fragments enriched in glycosylated peptides from the cytosolic fraction are treated with a glycosidase to release glycans.

21.-22. (canceled)

23. The method of claim 1, wherein said mass spectrometry is liquid chromatography-mass spectrometry.

24. The method of claim 1, wherein the profile of glycoproteins is obtained by searching the results of the mass spectrometry against a proteome database and a glycan database.

25. (canceled)

26. A method for detecting protein variation between samples comprising:
(a) processing a first sample comprising cells to isolate a cytosolic fraction of the cells and a membrane fraction of the cells;
(b) processing a second sample comprising cells to isolate a cytosolic fraction of the cells and a membrane fraction of the cells;
(c) digesting proteins in the cytosolic fraction and membrane fraction from step (a) to obtain cytosolic peptide fragments of the first sample;
(d) digesting proteins in the cytosolic fraction and membrane fraction from step (b) to obtain cytosolic peptide fragments of the second sample;
(e) labeling the cytosolic peptide fragments from the first sample with a first detectable marker and labeling the cytosolic peptide fragments from the second sample with a second detectable marker, and mixing the labeled cytosolic peptide fragments to obtain a mixture of labeled cytosolic peptide fragments from the first and second samples;
(f) labeling the membrane peptide fragments from the first sample with a third detectable label and labeling the membrane peptide fragments from the second sample with a fourth detectable label, and mixing the labeled membrane peptide fragments to obtain a mixture of labeled membrane peptide fragments from the first and second samples; and
(g) detecting the labeled cytosolic peptide fragments in the mixture of labeled cytosolic proteins from step (e); and detecting the labeled membrane peptide fragments in the mixture of labeled membrane proteins from step (f), thereby determining the variation in cytosolic proteins and variation in membrane proteins between the first sample and the second sample.

27. The method of claim 26, wherein
processing step (a) comprises:
(i) mixing the cells from the first sample with a permeabilization solution comprising a first detergent to permeabilize the plasma membrane of the cells in the first sample;
(ii) subjecting the mixture from step (i) to centrifugation to obtain a first pellet comprising permeabilized cells of the first sample, and a supernatant comprising the cytosolic fraction of the cells of the first sample;
(iii) collecting the supernatant from step (ii), and suspending the first pellet from step (ii) in a solubilization solution comprising a second detergent to form a suspension, wherein the second detergent solubilizes membrane proteins from the cells of the first sample;
(iv) subjecting the suspension from step (iii) to centrifugation to obtain a second pellet and a supernatant comprising the membrane fraction the cells of the first sample; and
(v) collecting the supernatant comprising the membrane fraction the cells of the first sample from step (iv), and
wherein processing step (b) comprises:
(aa) mixing the cells from the second sample with the permeabilization solution comprising the first detergent to permeabilize the plasma membrane of the cells in the second sample;
(bb) subjecting the mixture from step (aa) to centrifugation to obtain a pellet comprising permeabilized cells of the second sample, and a supernatant comprising the cytosolic fraction of the cells of the first sample;
(cc) collecting the supernatant from step (bb), and suspending the pellet from step (bb) in the solubilization solution comprising the second detergent to form a suspension, wherein the second detergent solubilizes membrane proteins from the cells of the second sample;
(dd) subjecting the suspension from step (cc) to centrifugation to obtain another pellet and a supernatant comprising the membrane fraction the cells of the second sample; and
(ee) collecting the supernatant comprising the membrane fraction the cells of the second sample from step (dd).

28. The method of claim 27, wherein the solubilization solution comprises an ionic detergent.

29. The method of claim 28, wherein the ionic detergent is selected from the group consisting of sodium dodecyl sulfate (SDS), sodium deoxycholate, N-lauryl sarcosine, 3-[(3-cholamidopropyl)dimethylammonio]-1-propanesulfonate (CHAPS) and a combination thereof.

30. The method of claim 28, wherein the solubilization solution comprises the ionic detergent at a concentration of 0.1% to 1.0% weight by volume.

31. The method of claim 27, the permeabilization solution comprises a nonionic detergent.

32. The method of claim 31, wherein the nonionic detergent is selected from the group consisting of Triton-X 100, octylphenoxypolyethoxyethanol, polysorbate 20 (Tween-20), Saponin and a combination thereof.

33. The method of claim 31, wherein the permeabilization solution comprises the nonionic detergent at a concentration of 0.1%-0.2% weight by volume.

34. The method of claim 26, further comprising performing a mass spectrometry analysis of the mixture of labeled cytosolic peptide fragments from step (e) to obtain a profile of glycoproteins in the cytosolic fractions of the first and second samples, and performing a mass spectrometry analysis the mixture of labeled membrane peptide fragments from step (f) to obtain a profile of glycoproteins in the membrane fractions of the first and second samples.

35. The method of claim 34, wherein the profile of glycoproteins in the cytosolic fraction of the first and second samples is obtained by a process comprising:
(1) prior to performing the mass spectrometry analysis, separating non-glycosylated peptide fragments from the mixture of labeled cytosolic peptide fragments to obtain collection sample of labeled cytosolic peptide fragments from the sample enriched in glycosylated peptides; and
(2) prior to step (g) performing a mass spectrometry analysis of the enriched sample of labeled cytosolic peptide fragments from the first and second samples obtained in step (1) to obtain the profile of glycoproteins in the cytosolic fractions from the first and second samples, wherein said profile comprises a listing of glycoproteins, optionally with one or more of glycosylation sites, glycopeptides, glycan composition, and abundance of the glycoproteins.

36. The method of claim 34, wherein the profile of glycoproteins in the membrane fraction of the first and second samples is obtained by a process comprising:
(1) prior to performing the mass spectrometry analysis, separating non-glycosylated peptide fragments from the mixture of labeled membrane peptide fragments to obtain collection sample of labeled membrane peptide fragments from the sample enriched in glycosylated peptides; and
(2) prior to step (g) performing a mass spectrometry analysis of the enriched sample of labeled membrane peptide fragments from the first and second samples obtained in step (1) to obtain the profile of glycoproteins in the membrane fractions from the first and second samples, wherein said profile comprises a listing of glycoproteins, optionally with one or more of glycosylation sites, glycopeptides, glycan composition, and abundance of the glycoproteins.

37. The method of claim 26, wherein the cells are mammalian cells.

38. (canceled)

39. The method of claim 27, wherein the first sample of step (a) and the second sample of step (b) are each a tissue sample, and the processing step (a) and (b) further comprise, prior to step (a)(i) and step (b)(aa) homogenizing each tissue sample.

40. (canceled)
